(19) United States
(12) Patent Application Publication
(10) Pub. No.: US 2024/0269170 A1
(43) Pub. Date: Aug. 15, 2024

(54) Treatment of Inflammatory Conditions or Diseases

(71) Applicant: Queen Mary University of London, London (GB)

(72) Inventors: Jesmond Dalli, London (GB); Kimberly Catherine De Freitas Pistorius, London (GB); Lucy Ly, London (GB)

(21) Appl. No.: 18/570,043

(22) PCT Filed: Jun. 14, 2022
(86) PCT No.: PCT/GB2022/051490

§ 371 Date: Dec. 13, 2023

(30) Foreign Application Priority Data
Jun. 14, 2021 (GB) 2108485.0

(51) Int. Cl. A61K 35/15; A61P 19/02; C12N 5/0786; G01N 33/92

(52) U.S. Cl. CPC A61K 35/15; A61P 19/02; C12N 5/0645; G01N 33/92; C12N 2501/999; G01N 2800/102

(57) ABSTRACT

The invention relates to agents and methods for treating or preventing inflammatory conditions or diseases.

### FIELD OF THE INVENTION

The present invention relates to specialized pro-resolving mediators (SPMs) or derivatives thereof for use in treating or preventing inflammatory conditions or diseases, in particular rheumatoid arthritis (RA). The invention also relates to cells that have been contacted with a specialized pro-resolving mediator (SPM) or derivative thereof, and their use in treating or preventing inflammatory conditions or diseases, in particular RA.

### BACKGROUND TO THE INVENTION

Rheumatoid arthritis (RA) is a progressive degenerative disorder that leads to joint destruction. To date, available treatments only target the inflammatory component with minimal impact on joint repair. Monocytes and macrophages play a central role in both disease propagation and resolution. Whilst much is known about factors that activate these cells to promote disease, little is known about the molecules and pathways that can be used to reprogram their responses to upregulate their anti-inflammatory and tissue-protective functions.

RA is a chronic inflammatory disorder characterized by dysregulated immune activation and unremitting inflammation. This persistent inflammatory response is linked with a progressive destruction of joints, leading to substantial morbidity and a reduction in quality of life.

The immune system plays a central role in both the propagation of joint inflammation as well as the observed tissue destruction. Among the immune cells known to participate in both the onset and termination of RA are monocytes. Findings made in experimental systems suggest that non-classical monocytes contribute to the onset of inflammatory arthritis, whereas classical monocytes are linked with the resolution of joint disease.

Progress has been made in the last few decades in both the early diagnosis and treatment of patients with RA. In particular, the development of biological drugs that target molecules linked with the propagation of arthritic inflammation, including tumour necrosis factor (TNF) and interleukin-6 (IL-6), has transformed the management of patients with this otherwise debilitating condition.

Despite the progress in the treatment of patients with RA, available therapeutics only target the inflammatory component of the disease without rectifying the extensive damage that occurs within the joints. Furthermore, a significant portion of patients build resistance to many of these biological drugs over time, limiting their effectiveness. Thus, there is a need for the development of therapeutics that not only limit arthritic inflammation but also promote joint repair.

### SUMMARY OF THE INVENTION

The present inventors have found that specialized pro-resolving mediators (SPMs) are effective in the treatment and prevention inflammatory conditions or diseases, in particular rheumatoid arthritis (RA). In particular, the inventors have found that maresin conjugate in tissue regeneration 3 (MCTR) not only successfully reduces joint inflammation in an RA model, but also leads to repair and protection of both bone and cartilage in joints.

The inventors have found that MCTR3 is effective both when administered directly to a subject and also when contacted with a culture of monocytes prior to the monocytes being administered to a subject.

While not wishing to be bound by theory, the inventors believe that MCTR3 may reprogram monocytes via the activation of epigenetic programs to confer enduring protective properties. Transcriptomic profiling and flow cytometric evaluation of monocyte-derived macrophages (MDM) from mice treated with MCTR3-reprogrammed monocytes revealed that Arginase-1 (Arg-1) may be involved in joint reparative and pro-resolving activities. The inventors' have shown that MCTR3 tempers mononuclear phagocytes responses, leading to the long-term upregulation of joint protective mechanisms during inflammatory arthritis.

In one aspect, the invention provides a specialized pro-resolving mediator (SPM) or derivative thereof for use in treating or preventing an inflammatory condition or disease.

In another aspect, the invention provides a cell for use in a method of treating or preventing an inflammatory condition or disease, wherein the method comprises the step of contacting a monocyte with a specialized pro-resolving mediator (SPM) or derivative thereof to obtain the cell.

In another aspect, the invention provides a cell for use in a method of treating or preventing an inflammatory condition or disease, wherein the cell has been contacted with a specialized pro-resolving mediator (SPM) or derivative thereof. The cell may be, for example, a monocyte or a cell differentiated therefrom, such as a monocyte-derived macrophage (MDM).

In another aspect, the invention provides a cell for use in a method of treating or preventing an inflammatory condition or disease, wherein the method comprises the step of contacting a monocyte-derived macrophage (MDM) with a specialized pro-resolving mediator (SPM) or derivative thereof to obtain the cell.

In some embodiments, the inflammatory condition or disease is rheumatoid arthritis (RA), osteoarthritis or atherosclerosis.

In preferred embodiments, the inflammatory condition or disease is RA.

In another aspect, the invention provides a specialized pro-resolving mediator (SPM) or derivative thereof for use in treating or preventing rheumatoid arthritis (RA).

In another aspect, the invention provides a cell for use in a method of treating or preventing rheumatoid arthritis (RA), wherein the method comprises the step of contacting a monocyte with a specialized pro-resolving mediator (SPM) or derivative thereof to obtain the cell.

In another aspect, the invention provides a cell for use in a method of treating or preventing rheumatoid arthritis (RA), wherein the cell has been contacted with a specialized pro-resolving mediator (SPM) or derivative thereof. The cell may be, for example, a monocyte or a cell differentiated therefrom, such as a monocyte-derived macrophage (MDM).

In another aspect, the invention provides a cell for use in a method of treating or preventing rheumatoid arthritis (RA), wherein the method comprises the step of contacting a monocyte-derived macrophage (MDM) with a specialized pro-resolving mediator (SPM) or derivative thereof to obtain the cell.

In some embodiments, the SPM is selected from the group consisting of a maresin, a protectin, a resolvin and an E-series resolvin. In some embodiments, the SPM is a maresin.

In some embodiments, the SPM is a maresin conjugate in tissue regeneration (MCTR).

In some embodiments, the SPM is selected from the group consisting of MCTR3, MCTR1 and MCTR2.

In preferred embodiments, the SPM is MCTR3. In some embodiments, the SPM is MCTR2. In some embodiments, the SPM is MCTR1.

In some embodiments, the cell is a monocyte-derived macrophage (MDM).

In some embodiments, the treatment of RA reduces joint inflammation, and/or repairs bone and/or cartilage. In some embodiments, the treatment of RA reduces joint inflammation. In preferred embodiments, the treatment of RA repairs bone and/or cartilage.

In some embodiments, joint inflammation is reduced or prevented. In some embodiments, bone and/or cartilage is repaired or protected.

In some embodiments, joint oedema is reduced. In some embodiments, bone and/or cartilage turnover is reduced. In some embodiments, bone volume is increased. In some embodiments, cartilage volume is increased. In some embodiments, leukocyte infiltration is decreased.

In some embodiments, the SPM is MCTR3 and the treatment of RA reduces joint inflammation, and/or repairs bone and/or cartilage. In some embodiments, the SPM is MCTR3 and the treatment of RA reduces joint inflammation. In preferred embodiments, the SPM is MCTR3 and the treatment of RA repairs bone and/or cartilage.

In some embodiments, the SPM is MCTR3 and joint inflammation is reduced or prevented. In some embodiments, the SPM is MCTR3 and bone and/or cartilage is repaired or protected.

In some embodiments, the SPM is MCTR3 and joint oedema is reduced. In some embodiments, the SPM is MCTR3 and bone and/or cartilage turnover is reduced. In some embodiments, the SPM is MCTR3 and bone volume is increased. In some embodiments, the SPM is MCTR3 and cartilage volume is increased. In some embodiments, the SPM is MCTR3 and leukocyte infiltration is decreased.

In some embodiments, the SPM or derivative thereof or cell is administered to a subject after RA onset.

In some embodiments, the SPM or derivative thereof or cell is administered to a subject after failure of DMARD treatment.

In some embodiments, the SPM or derivative thereof or cell is administered during arthritic inflammation.

In some embodiments, the SPM or derivative thereof or cell is administered via intravenously or intra-articularly.

In another aspect, the invention provides a method of culturing a population of monocytes, the method comprising:
(a) providing a population of monocytes; and
(b) contacting the population of monocytes with a specialized pro-resolving mediator (SPM) or derivative thereof.

In some embodiments, the monocytes are reprogrammed.

In another aspect, the invention provides a method of reprogramming monocytes, the method comprising:
(a) providing a population of monocytes; and
(b) contacting the population of monocytes with a specialized pro-resolving mediator (SPM) or derivative thereof.

In another aspect, the invention provides a method of culturing a population of monocyte-derived macrophages (MDMs), the method comprising:
(a) providing a population of MDMs; and
(b) contacting the population of MDMs with a specialized pro-resolving mediator (SPM) or derivative thereof.

In some embodiments, the SPM is selected from the group consisting of a maresin, a protectin, a resolvin and an E-series resolvin. In some embodiments, the SPM is a maresin.

In some embodiments, the SPM is a maresin conjugate in tissue regeneration (MCTR).

In some embodiments, the SPM is selected from the group consisting of MCTR3, MCTR 1 and MCTR2.

In preferred embodiments, the SPM is MCTR3. In some embodiments, the SPM is MCTR2.

In some embodiments, the SPM is MCTR1.

In some embodiments, monocytes are differentiated to monocyte-derived macrophages (MDMs). In some embodiments, the population of monocytes is contacted with one or more growth factor. In some embodiments, the population of monocytes is contacted with M-CSF or GM-CSF. In some embodiments, the population of monocytes is contacted with serum, for example FBS. In some embodiments, the population of monocytes is contacted with the growth factor and the serum. In some embodiments, the population of monocytes is contacted with the growth factor and/or serum for 1-7 days.

In some embodiments, the population is contacted with the SPM or derivative thereof for between 1 hour and 10 days, for example between 2 hours and 10 days, between 12 hours and 10 days, between 1 and 10 days, between 2 and 10 days, or between 2 and 7 days.

In some embodiments, the population is contacted with the SPM or derivative thereof for 1-72 hours, for example 1-48 hours, 1-24 hours, 1-12 hours or 2-12 hours.

In some embodiments, the population is contacted with the SPM or derivative thereof for 2-12 hours.

In some embodiments, the population is contacted with the SPM or derivative thereof before the contact with the growth factor and/or serum. In some embodiments, the population is contacted with the SPM or derivative thereof before and during the contact with the growth factor and/or serum.

In some embodiments, the population is washed after the contact with the SPM or derivative thereof and before the contact with the growth factor and/or serum.

In some embodiments, the SPM or derivative thereof is contacted with the population of monocytes at a final concentration of 0.001-100 nM, for example 0.01-100 nM, 0.1-100 nM, 0.1-10 nM, 0.1-5 nM, 0.5-5 nM, 0.5-4 nM, 0.5-3 nM or 0.5-2 nM. In some embodiments, the SPM or derivative thereof is contacted with the population of monocytes at a final concentration of about 0.1 nM, 0.2 nM, 0.3 nM, 0.4 nM, 0.5 nM, 0.6 nM, 0.7 nM, 0.8 nM, 0.9 nM, 1 nM, 2 nM, 3 nM, 4 nM, 5 nM, 6 nM, 7 nM, 8 nM, 9 nM, 10 nM, 25 nM, 50 nM, 75 nM, or 100 nM.

In another aspect, the invention provides a population of cells obtainable by the method of the invention. The population of cells may be, for example, comprise monocytes or cells differentiated therefrom, such as monocyte-derived macrophages (MDMs).

In another aspect, the invention provides a pharmaceutical composition comprising the population of cells of the invention and a pharmaceutically-acceptable carrier, excipient and/or diluent.

In another aspect, the invention provides the population of cells or pharmaceutical composition of the invention for use in treating or preventing an inflammatory condition or disease.

In some embodiments, the inflammatory condition or disease is rheumatoid arthritis (RA), osteoarthritis or atherosclerosis.

In preferred embodiments, the inflammatory condition or disease is RA.

In another aspect, the invention provides the population of cells or pharmaceutical composition of the invention for use in treating or preventing rheumatoid arthritis (RA).

In some embodiments, the contacting with the SPM or derivative thereof increases expression of arginase-1 (Arg-1), interleukin-10 (IL-10), Dbl1 and/or TGFb.

In some embodiments, the cells are Arg1+, IL-10+, DBI1+ and/or TGFb+.

In another aspect, the invention provides a cell, wherein the cell has increased expression of arginase-1 (Arg-1), interleukin-10 (IL-10), Dbl1 and/or TGFb in comparison to an otherwise substantially identical cell that has not been contacted with the specialized pro-resolving mediator (SPM) or derivative thereof. The cell may be, for example, a monocyte or a cell differentiated therefrom, such as a monocyte-derived macrophage (MDM).

In another aspect, the invention provides a method of treating or preventing an inflammatory condition or disease comprising administering a specialized pro-resolving mediator (SPM) or derivative thereof to a subject in need thereof.

In another aspect, the invention provides a method of treating or preventing an inflammatory condition or disease comprising the steps:
(a) providing a population of monocytes;
(b) contacting the population of monocytes with a specialized pro-resolving mediator (SPM) or derivative thereof; and
(c) administering the cells from step (b) or a portion thereof to a subject in need thereof.

In another aspect, the invention provides a method of treating or preventing an inflammatory condition or disease comprising the steps:
(a) providing a population of monocyte-derived macrophages (MDMs);
(b) contacting the population of MDMs with a specialized pro-resolving mediator (SPM) or derivative thereof; and
(c) administering the cells from step (b) or a portion thereof to a subject in need thereof.

In some embodiments, the inflammatory condition or disease is rheumatoid arthritis (RA), osteoarthritis or atherosclerosis.

In preferred embodiments, the inflammatory condition or disease is RA.

In another aspect, the invention provides a method of treating or preventing rheumatoid arthritis (RA) comprising administering a specialized pro-resolving mediator (SPM) or derivative thereof to a subject in need thereof.

In another aspect, the invention provides a method of treating or preventing rheumatoid arthritis (RA) comprising the steps:
(a) providing a population of monocytes;
(b) contacting the population of monocytes with a specialized pro-resolving mediator (SPM) or derivative thereof; and
(c) administering the cells from step (b) or a portion thereof to a subject in need thereof.

In another aspect, the invention provides a method of treating or preventing rheumatoid arthritis (RA) comprising the steps:
(a) providing a population of monocyte-derived macrophages (MDMs);
(b) contacting the population of MDMs with a specialized pro-resolving mediator (SPM) or derivative thereof; and
(c) administering the cells from step (b) or a portion thereof to a subject in need thereof.

In another aspect, the invention provides a method of diagnosing an inflammatory condition or disease comprising the steps:
(a) determining the level of one or more maresin conjugate in tissue regeneration (MCTR) in one or more sample obtained from a subject;
(b) comparing the level of the one or more MCTR to one or more corresponding reference value;
   wherein the level of the one or more MCTR compared to the one or more reference value is indicative of the inflammatory condition or disease.

In some embodiments, the inflammatory condition or disease is rheumatoid arthritis (RA), osteoarthritis or atherosclerosis.

In preferred embodiments, the inflammatory condition or disease is RA.

In another aspect, the invention provides a method of diagnosing rheumatoid arthritis (RA) comprising the steps:
(a) determining the level of one or more maresin conjugate in tissue regeneration (MCTR) in one or more sample obtained from a subject;
(b) comparing the level of the one or more MCTR to one or more corresponding reference value;
   wherein the level of the one or more MCTR compared to the one or more reference value is indicative of RA.

In some embodiments, the sample is a plasma sample.

In some embodiments, the MCTR is selected from the group consisting of MCTR3, MCTR1 and MCTR2. In preferred embodiments, the MCTR is MCTR3.

### DESCRIPTION OF THE DRAWINGS

FIG. 1: MCTR3 displays anti-arthritic activity in inflammatory arthritis. (A-D) K/B×N serum (100 μL via intraperitoneal injection, i.p.) was administered to C57BL/6 mice on days 0, 2 and 8 to initiate and prolong inflammatory arthritis. Mice were treated 1 μg/mouse MCTR3 or vehicle (PBS+0.1% EtOH) on day 10, 12 and 14 via intravenous (i.v.) injection. Disease course was assessed by evaluating A) clinical scores and B) paw oedema daily. Results are mean±SEM, n=9 mice per group. Statistical differences were evaluated using a Two-Way ANOVA. Arrows denote days when MCTR3 was administered. (C,D) paws were harvested on day 25, and cells liberated from the joints and the expression of phenotypic markers was evaluated on joint macrophages using flow cytometry and PLS-DA. (C) scores plot with highlighted regions denoting the clusters representing cells from each group and (D) VIP scores for each of the markers evaluated. Each dot in the score plot represents a separate mouse. (E-H) Arthritis was induced in DBA/1 mice by administration of 10 μg G6PI in CFA on Day 0. On day 24, 26 and 28 (denoted in blue arrows), mice were administered 1 μg/mouse MCTR3 or vehicle (PBS+0.1% EtOH) i.v. and (E) clinical scores and (F) paw oedema were measured. Results are mean±SEM, n=mice 7 per group from 2 separate experiments. Statistical differences were evaluated using a Two-Way ANOVA. (G-H) paws were harvested on day 36, and cells liberated from the joints and the expression of phenotypic markers was evaluated on joint macrophages using flow cytometry and PLS-DA. (G) scores plot with highlighted regions denoting the clusters representing cells from each group and (H) VIP scores for each of the markers evaluated. Each dot in the score plot represents a separate mouse.

FIG. 2: MCTR3 promotes bone and cartilage repair. C57BL/6 mice were administered 100 μL K/B×N serum i.p. on day 0, 2 and 8. Mice were treated with 1 μg/mouse MCTR3 or vehicle (PBS+0.1% EtOH) i.v. on day 10, 12 and 14. Hind paws and blood were collected on day 25 and A) glycosaminoglycan content was evaluated using Safranin-O staining. (left and centre panels) Representative images from Safranin-O stained knee joints and (right panel) quantification of Safranin-O stained knee joints. Results are mean±SEM, n=8 per group. Statistical differences were evaluated using Mann-Whitney U test. B-D) Expression of collagen type 2 (Col 2) and collagen type X (Col X) was evaluated using immunohistochemistry. B) Representative images from immunofluorescent assessment of Col 2 and Col X expression, C) mean intensity of Col 2 and D) average number of cells expressing Col X. Results are mean±SEM, n=5 per group, statistical differences were evaluated using Mann-Whitney U test. E-I) microCT analysis was performed on ankle joints ex vivo to assess bone callus cover. D) Representative images of ankles from vehicle and MCTR3 treated mice, where red represents the callus area. F) Callus tissue volume, G) bone volume, H) bone surface and I) the ratio of bone surface to bone volume of the ankles from mice treated with either vehicle or MCTR3. Results are mean±SEM, n=8 per group from two separate experiments. Statistical differences were evaluated Mann-Whitney U test. J,K) Blood was collected at the end of the experiment and plasma PINP and CTX-I levels were assessed. Results are mean±SEM, n=5 for Vehicle and n=7 for MCTR3 groups. Statistical differences were evaluated Mann Whitney U test.

FIG. 3: MCTR3 reprograms monocyte responses to reduce inflammation and repair inflamed joints. (A-B) Antigen induced arthritis was initiated by administering G6PI peptide (10 μg/mouse) in CFA via intradermal injection to DBA/1 mice. After 25 days mice were treated with 0.5×10⁶ monocytes from naive mice that had been pre-incubated with vehicle (PBS+0.1% EtOH) or 1 nM MCTR3 for 90 min at 37° C. via i.v. injection. Disease course was monitored daily by assessing A) clinical scores and B) oedema. Results are mean±SEM and expressed as percent change from day of treatment n=5 per group from two separate experiments. Results are mean±SEM. Statistical differences were calculated using a Two-way ANOVA.

(C-D) K/B×N serum (100 μL, i.p.) was administered to C57BL/6 mice on day 0, 2 and 9 to induce and prolong inflammatory arthritis and, on day 12, mice were treated i.v. with 2×10⁶ monocytes isolated from arthritic mice and incubated with either vehicle (PBS+0.1% EtOH) or 1 nM MCTR3 for 90 min at 37° C. Disease course was monitored daily by assessing C) clinical scores and D) oedema. Results are mean±SEM. and expressed as percent change from day of treatment. n=9 per group from two distinct experiments. (E) On day 22 hind paws were harvested joints were fixed, sectioned, stained using H&E stain and leukocyte infiltration evaluated. Top and centre panels present representative images from each experimental group; bottom panel Quantitation of the scores in each of the group. Results are mean±SEM. n=8 mice per group. Statistical differences were evaluated using Mann-Whitney U test. IFP=intrapatellar fat, M=meniscus, TB=Tibia, PF=Pannus formation, arrows denote leukocyte infiltration. (F-G) Paws were harvested 10 days after treatment and lipid mediator profiles were determined using LC-MS/MS-based lipid mediator profiling and evaluated using PLS-DA. (F) scores plot with highlighted regions denoting the clusters representing cells from each group (G) VIP scores for top 15 mediators. Each dot in the score plot represents a separate mouse.

FIG. 4: Regulation of joint reparative pathways in mice receiving MCTR3-trained monocytes. K/B×N serum (100 μL, i.p.) was administered to C57BL/6 mice on day 0, 2 and 9 to induce and prolong inflammatory arthritis and, on day 12, mice were treated i.v. with 2×10⁶ monocytes isolated from arthritic mice and incubated with either vehicle (PBS+0.1% EtOH) or 1 nM MCTR3 for 90 min. (A-C) On day 22 hind paws were harvested, fixed, and stained to evaluate A) proteoglycan content using Safranin-O staining; B) Col 2 and C) Col X expression was evaluated using immunofluorescence. Left panels present representative images from each experimental group, right panels provide a quantitative evaluation of the staining. Results are expressed as percent change vs Monocyte group. n=7-8 mice per group. Statistical differences were evaluated using Wilcoxon signed rank test. (D-I) Hind paws were also collected on day 22 to evaluate the expression of the indicated genes was evaluated using quantitative realtime PCR. Results are from n=5 mice per group. Statistical differences were evaluated using Mann Whitney-U test.

FIG. 5: MCTR3 reprograms the arthritic monocyte-derive macrophage transcriptome. A-B) Arthritis was induced and prolonged in C57BL/6 mice by administering 100 μL K/B×N serum i.p. on day 0, 2 and 9. Mice were treated on day 12 i.v. with 2×10⁶ monocytes isolated from arthritic mice and incubated either with vehicle (PBS+0.1% DMSO) or 10 μM RG108, a DNMT inhibitor, for 15 min and then with a vehicle (PBS+0.1% EtOH) or 1 nM MCTR3 for 90 min (37° C.). Disease course was monitored daily by assessing A) clinical scores and B) oedema. Results are mean±SEM and expressed as percent change from day of treatment. n=10 mice per group. Statistical differences were evaluated using Two-way ANOVA. (C-E) K/B×N serum (100 μL, i.p.) was administered to mice on days 0, 2 and 9 to induce and prolong inflammatory arthritis. On day 12, mice were treated i.v. with 2×10⁶ monocytes isolated from arthritic mice that were previously incubated with either vehicle (PBS+0.1% EtOH) or 1 nM MCTR3 for 90 min. Cells were isolated from paw joints on day 22, sorted for CD45⁺ cells and single cell RNA sequencing was performed. C) t-SNE layout of clusters obtained from isolated leukocyte populations, D) volcano plot highlighted differentially regulated genes and E) the gene network analysis for genes that were found to be differentially regulated in MDM isolated from mice receiving MCTR3-reprogrammed monocytes when compared with MDM receiving monocytes incubated with vehicle. Results are from n=4 mice per group. (F-G) Monocytes were isolated from human healthy volunteers, these cells were then incubated with GM-CSF (7 days, 37° C.) then with either Vehicle or MCTR3 (1 nM, 37° C.). Cells were lysed and the phosphoproteome determined using mass spectrometry. (F) GO Biological pathway analysis and (G) KEGG pathway analysis for proteins found to be differentially phosphorylated in cells incubated with MCTR3 when compared to those incubated with vehicle. Results are representative of cells from n=3 healthy volunteers per group

FIG. 6: Arg-1 mediates the anti-inflammatory and cartilage protective activities of MCTR3-trained monocytes. (A,B) Femur heads and bone marrow-derived monocytes were collected 5 days following the induction of arthritis in C57BL/6 mice. Femur heads were incubated in serum free DMEM high glucose containing 1% insulin-transferrin-selenium for 48 hours and then in DMEM containing 10% FBS and 10 ng/ml IL-1B for 4 days. Monocytes were incubated with vehicle (PBS+0.1% EtOH) or 1 nM MCTR3 for 24 hours, then differentiated to monocyte-derived macrophages. Two days after the initiation of differentiation, cells were transfected with control siRNA or siRNA against Arg-1. Three days later these cells were incubated with arthritic femur heads for 48 hours. Tissues were then collected and glycosaminoglycan content was evaluated using Safranin-O staining. (A) Representative images and (B) quantitation of Safranin-O staining. Results are mean±SEM, n=7-8 mice per group per group. Statistical differences were evaluated using Wilcoxon signed rank test when assessing for differences vs Vehicle group and using one-way ANOVA and Kruskal Wallis post hoc test when evaluating differences vs MCTR3 treated group.

(C-F) Mice were administered K/B×N serum on days 0, 2 and 9 then on day 12 they were treated 2×10⁶ PKH67-labelled monocytes isolated from arthritic mice and incubated with either vehicle (PBS+0.1% EtOH) or 1 nM MCTR3 for 90 min via i.v injection and 200 μg N^{ω}-Hydroxy-nor-L-arginine (nor-NOHA), an arginase 1 inhibitor, or vehicle (PBS) that were administered daily for a 10-day period via i.p. injection. Disease course was evaluated by assessing (C) clinical scores and (D) paw oedema. Results are mean±SEM, n=8-10 per group from two distinct experiments and expressed as percent change from first day of treatment. Statistical differences were evaluated using Two-way ANOVA. E-F) At the end of the experiments joints were collected and E) Dbl and F) TGF-β1 expression was evaluated in PKH67⁺ CD64⁺ cells using flow cytometry. Results are mean±SEM and expressed as percentage change from vehicle group. n=9-10 mice per group. Statistical differences were evaluated using Wilcoxon signed rank test when assessing for differences vs Vehicle group and using one-way ANOVA and Kruskal Wallis post hoc test when evaluating differences vs MCTR3 treated group.

FIG. 7: MCTR3 limits joint inflammation. Mice were administered K/B×N serum on days 0 and 2 and then treated with MCTR3 (100 ng/mouse) or Vehicle (PBS+0.1% EtOH) on days 3 and 5. Disease course was evaluated by assessing (A) clinical scores. (B-C) On day 7 joints were collected and disease severity was evaluated using (B) H&E Staining and (C) glycosaminoglycan content was assessed using Safranin-O staining, arrows denote Safranin-O staining in cartilage. Results for A are presented as mean±SEM. Statistical differences for A were evaluated using Two-Way ANOVA. n=4 mice per group. IFP=intrapatellar fat, M=meniscus, TB=Tibia, PF=Pannus formation, Red arrows denote leukocyte infiltration. (D-E) Lipid mediator profiles were determined using LC-MS/MS-based lipid mediator profiling and evaluated using PLS-DA. (D) scores plot with highlighted regions denoting the clusters representing cells from each group and (E) VIP scores for top 15 mediators. Each dot in the score plot represents a separate mouse.

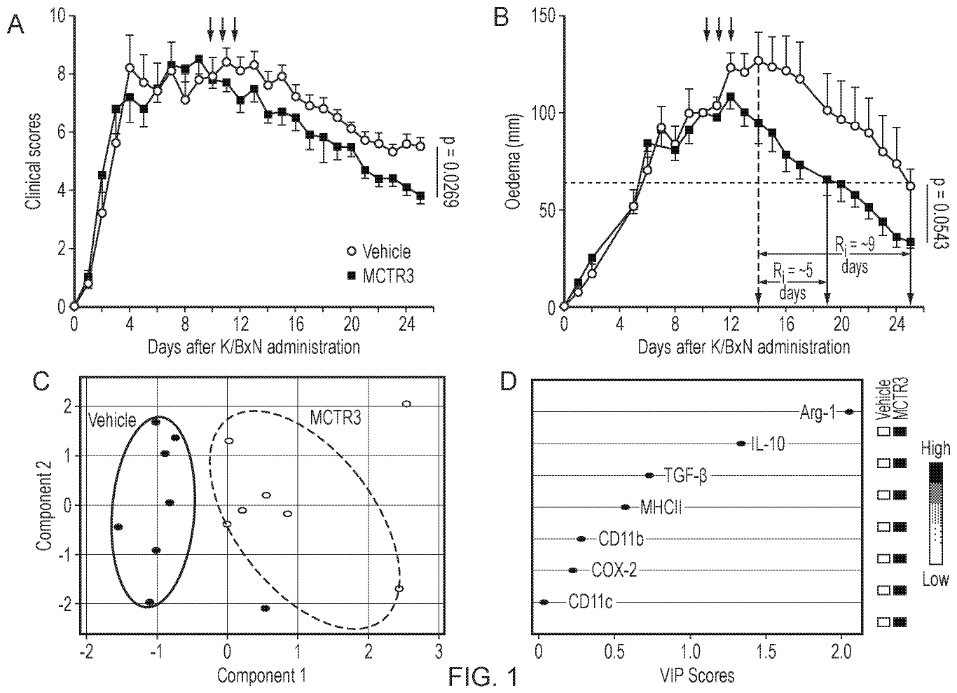

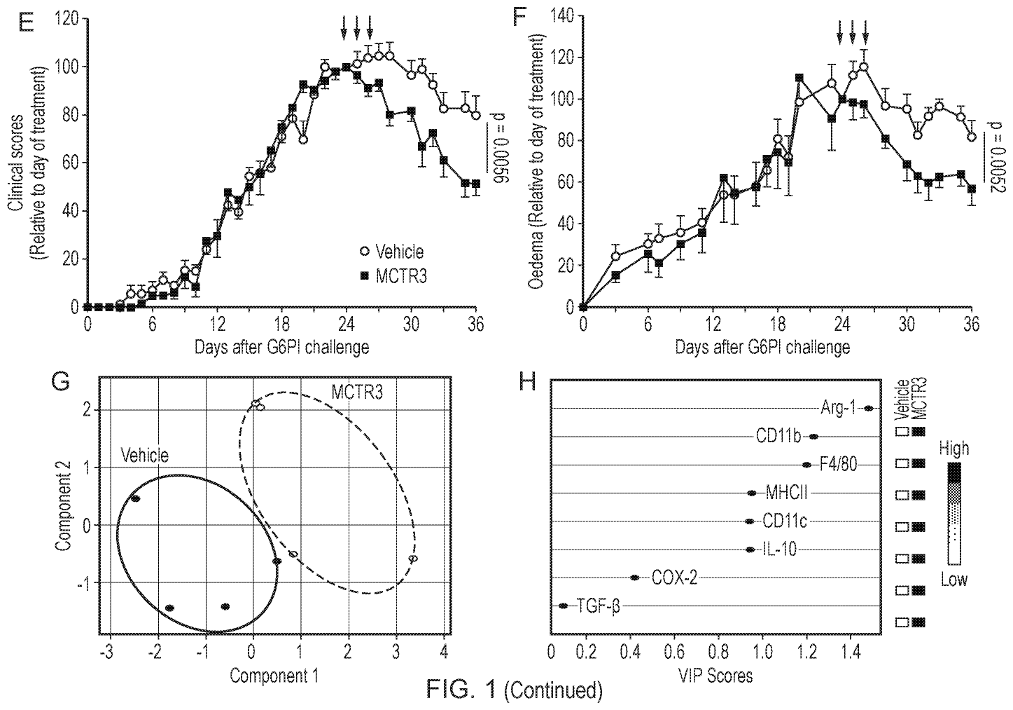

FIG. 8: MCTR3 decreased leukocyte infiltration and cartilage damage in arthritic mice. Related to FIG. 1. C57BL/6 mice were administered 100 μL K/B×N serum i.p. on day 0, 2 and 8. Mice were treated with 1 μg/mouse MCTR3 or vehicle i.v. (PBS+0.1% EtOH) on day 10, 12 and 14. Hind paws were collected on day 25, fixed and stained using H&E stain. (A) representative images (B) Pannus size (C) leukocyte infiltration and (D) cartilage damage were evaluated. Results are mean±SEM. n=5 mice per group. IFP=intrapatellar fat, M=meniscus, TB=Tibia, PF=Pannus formation, Red arrows denote leukocyte infiltration.

FIG. 9: Gating strategy employed in the identification of monocyte-derived macrophages.

FIG. 10: MCTR3 promotes bone and cartilage repair in arthritic mice. G6PI peptide (10 μg/mouse) in CFA was administered to DBA/1 mice (120 L/mouse) and mice were treated with either vehicle (PBS+0.1% EtOH) or 1 μg/mouse MCTR3 i.v. on day 24, 26 and 28. On day 36, hind paws were harvested for histological analysis. A) Representative images from knee joints from a vehicle and MCTR3 treated mouse stained with Safranin-O and B) quantitative analysis of Safranin O staining. Results are mean±SEM n=5 mice per group. C-D) microCT analysis was performed in vivo on day 24 and 35, to evaluate bone erosion in the arthritic knees. C) Representative images from the microCT scans from the knee from arthritic mice treated with either vehicle or MCTR3 exhibiting a 2-D coronal cross-sectional image of the proximal tibia, where the ROI was selected in the trabecular epiphysis and D) quantitative analysis of bone volume. Results are mean±SEM and expressed as percent change from values obtained on day 24 for each mouse. n=4 mice per group. Statistical differences were evaluated using a Mann-Whitney U test.

FIG. 11: DNA methyltransferase inhibition prevents MCTR3 reprogramming of protective monocyte responses. Arthritis was induced in DBA/1 mice by intradermal administration of 10 μg G6PI in CFA (120 μL/mouse) and on day 25, mice were treated via i.v. injection with 0.5×10⁶ monocytes from naive mice that had been pre-incubated with vehicle (PBS+0.1% DMSO) or 10 μM RG108, a DNMT inhibitor, for 15 min and then with a vehicle (PBS+0.1% EtOH) or 1 nM MCTR3 for 90 min at 37° C. Disease course was assessed by evaluating A) clinical scores and B) oedema relative to the day of treatment. Results are mean±SEM and expressed as percentage change from day of treatment. n=5 per group and statistics were calculated using a Two-way ANOVA.

FIG. 12: DNMT enzyme inhibition reverses the ability of MCTR3 to upregulate Arg-1 in MDM and the cartilage protective activities of MCTR3-trained monocytes. (A) Inflammatory arthritis was induced in C57BL/6 mice by administering 100 μL K/B×N serum i.p. on days 0 and 2 and femur heads and bone marrow monocytes were collected on day 5. Femur heads were incubated in serum free DMEM high glucose containing 1% insulin-transferrin-selenium for 48 hours and then in DMEM containing 10% FBS and 10 ng/ml IL1-β for a further 72 hours. Bone marrow derived monocytes were incubated with vehicle (PBS+0.1% DMSO) or 10 μM RG108, a DNMT inhibitor, for 45 min and then with either vehicle (PBS+0.1% EtOH) or 1 nM MCTR3 for 24 hours. Cells were then differentiated to monocyte-derived macrophages, then incubated with femur heads for 48 hours. The proteoglycan concentrations in the femur heads were assessed using Safranin O staining. (left panels) Representative images from Safranin-O stained knee joints and (right panel) quantification of Safranin-O stained knee joints. Results are mean±sem and expressed as percentage change from cells incubated with vehicle alone. n=5-8 mice per group from two separate experiments. Statistical differences were evaluated using one-sample Wilcoxon signed rank test.

(B) Mice were administered K/B×N serum on days 0, 2 and 9 then on day 12 they were treated 2×10⁶ PKH67-labelled monocytes isolated from arthritic mice and incubated with either vehicle (PBS+0.1% EtOH) or 1 nM MCTR3 for 90 min via i.v injection. After 10 days joints were harvested, cells were liberated that the expression of Arg-1 was evaluated in PKH67⁺CD64⁺ cells using flow cytometry. Results are from n=10 mice per group. Statistical differences were evaluated using a Mann-Whitney U test. (C) Inflammatory arthritis was induced in C57BL/6 mice and bone marrow-derived monocytes were isolated and treated as in A and Arg-1 expression was evaluated using flow cytometry. Results are mean±SEM and expressed as percentage change from cells incubated with vehicle alone. n=8 per group from two separate experiments. Statistical differences were evaluated using one-sample Wilcoxon signed rank test. (D) Mice were given K/B×N serum (via i.p. injection) on days 0, 2 and 9. On day 12 these were treated with 2×10⁶ PKH67-labelled monocytes isolated from arthritic mice and incubated either with vehicle (PBS+0.1% DMSO) or 10 μM RG108, a DNMT inhibitor, for 15 min and then with a vehicle (PBS+0.1% EtOH) or 1 nM MCTR3 for 90 min. On day 22, joints were collected and the expression of Arg-1 in PKH67⁺ CD64⁺ cells was evaluated using flow cytometry. Results are mean±SEM and expressed as percentage change from vehicle group. n=5 mice per group. Statistics differences were evaluated Mann-Whitney U test. Dashed line represents Vehicle groups.

### DETAILED DESCRIPTION OF THE INVENTION

The terms “comprising”, “comprises” and “comprised of” as used herein are synonymous with “including” or “includes”; or “containing” or “contains”, and are inclusive or open-ended and do not exclude additional, non-recited members, elements or steps. The terms “comprising”, “comprises” and “comprised of” also include the term “consisting of”.

### Inflammatory Conditions

The present invention relates to specialized pro-resolving mediators (SPMs) or derivatives thereof and their use in treating or preventing inflammatory conditions or diseases. The invention also relates to cells that have been contacted with a specialized pro-resolving mediator (SPM) or derivative thereof, and their use in treating or preventing inflammatory conditions or diseases.

In some embodiments, the inflammatory condition or disease is inflammatory arthritis.

In some embodiments, the inflammatory condition or disease is rheumatoid arthritis (RA), osteoarthritis or atherosclerosis.

In preferred embodiments, the inflammatory condition or disease is RA.

### Rheumatoid Arthritis (RA)

Rheumatoid arthritis (RA) is a chronic, systemic inflammatory disorder that may affect many tissues and organs, but principally attacks synovial joints. It is a disabling and painful condition, which can lead to substantial loss of functioning and mobility if not adequately treated.

The disease process involves an inflammatory response of the synovium, secondary to massive immune cell infiltration and proliferation of synovial cells, excess synovial fluid, and the development of fibrous tissue (pannus) in the synovium that attacks the cartilage and sub-chondral bone. This often leads to the destruction of articular cartilage and the formation of bone erosions with secondary ankylosis (fusion) of the joints. RA can also produce diffuse inflammation in the lungs, the pericardium, the pleura, the sclera, and also nodular lesions, most commonly in subcutaneous tissue. RA is considered a systemic autoimmune disease as autoimmunity plays a pivotal role in its chronicity and progression.

A number of cell types are involved in the aetiology of RA, including T cells, B cells, monocytes, macrophages, dendritic cells and synovial fibroblasts. Autoantibodies known to be associated with RA include those targeting Rheumatoid factor (RF) and anti-citrullinated protein antibodies (ACPA).

### Specialized Pro-Resolving Mediators (SPMs)

Specialized pro-resolving mediators (SPMs) are molecules produced by enzymes primarily carried in leukocytes that may act on the essential fatty acids arachidonic acid (AA), eicosapentaenoic acid (EPA), n-3 docosapentaenoic acid (n-3 DPA) and docosahexaenoic acids (DHA).

The SPM may, for example, be a DHA metabolite, n-3 DPA metabolite, AA metabolite or an EPA metabolite.

In some embodiments, the SPM is a DHA metabolite or an EPA metabolite.

### EPA Metabolites

EPA metabolites include the “E-series resolvins” and EPA-derived monohydroxylated fatty acids

Example EPA metabolites are listed in Table 1 below:

TABLE 1

| Abbreviation | Trivial name | Full stereochemistry |
|---|---|---|
| RvE1 | Resolvin E1 | 5S, 12R, 18R-trihydroxy-eicosa-6Z, 8E, 10E, 14Z, 16E- |
| | | pentaenoic acid |
| RvE2 | Resolvin E2 | 5S, 18R-dihydroxy-eicosa-6Z, 8E, 11E, 14E, 16Z- |
| | | pentaenoic acid |
| RvE3 | Resolvin E3 | 17R, 18R/S-dihydroxy-eicosa-5Z, 8Z, 11Z, 13E, 15E- |
| | | pentaenoic acid |
| 18-HEPE | | 18-hydroxy-eicosa-5Z, 8Z, 11Z, 14Z, 16E-pentaenoic acid |
| 15-HEPE | | 15-hydroxy-eicosa-5Z, 8Z, 11Z, 13E, 17Z-pentaenoic acid |
| 12-HEPE | | 12-hydroxy-eicosa-5Z, 8Z, 10E, 14Z, 17Z-pentaenoic acid |
| 5-HEPE | | 5-hydroxy-eicosa-6E, 8Z, 11Z, 14Z, 17Z-pentaenoic acid |

### n-3 DPA Metabolites

n-3 DPA metabolites include the 13-series resolvins—RvT1, RvT2, RvT3 and RvT4, D-series resolvins—RvD1_{n-3 DPA}, RvD2_{n-3 DPA} and RvD5_{n-3 DPA}, Protectins PD1_{n-3 DPA} PD2_{n-3 DPA} and 10S, 17S-diHDPA and Maresins—MaR1_{n-3 DPA} MAR2_{n-3 DPA} and 7S, 14S-diHDPA together with the respective monohydroxylated fatty acids.

Example n-3 DPA metabolites are listed in Table 2 below:

TABLE 2

| Trivial | | |
|---|---|---|
| name | Abbreviation | Full stereochemistry |
| Resolvin T1 | RvT1 | 7, 13R, 20-trihydroxy-docosa-8E, 10Z, 14E, 16Z, 18E- |
| | | pentaenoic acid |
| Resolvin T2 | RvT2 | 7, 12, 13R-trihydroxy-docosa-8Z, 10E, 14E, 16Z, 19Z- |
| | | pentaenoic acid |
| Resolvin T3 | RvT3 | 7, 8, 13R-trihydroxy-docosa-9E, 11E, 14E, 16Z, 19Z- |
| | | pentaenoic acid |
| Resolvin T4 | RvT4 | 7, 13R-dihydroxy-docosa-8, 10Z, 14E, 16Z, 19Z- |
| | | pentaenoic acid |
| Resolvin | RvD1_{n-3 DPA} | 7S, 8R, 17 S-trihydroxy-9E, 11E, 13Z, 15E, 19Z- |
| D1_{n-3 DPA} | | docosapentaenoic acid |
| Resolvin | RvD2_{n-3 DPA} | 7S, 16R, 17 S-trihydroxy-8E, 10Z, 12E, 14E, 19Z- |
| D2_{n-3 DPA} | | docosapentaenoic acid |
| Resolvin | RvD5_{n-3DPA} | 7S, 17S-dihydroxy-8E, 10Z, 13Z, 15E, 19Z- |
| D5_{n-3 DPA} | | docosapentaenoic acid |
| Protectin | PD1_{n-3 DPA} | 10R, 17S-dihydroxy-7Z, 11E, 13E, 15Z, 19Z- |
| D1_{n-3 DPA} | | docosapentaenoic acid |
| Protectin | PD2_{n-3 DPA} | 16R, 17S-dihydroxy-7Z, 10Z, 12E, 14E, 19Z- |
| D2_{n-3 DPA} | | docosapentaenoic acid |
| 10S, 17S- | 10S, 17S- | 10S, 17S-dihydroxy-7Z, 11E, 13Z, 15E, 19Z- |
| diHDPA | diHDPA | docosapentaenoic acid |
| Maresin | MaR 1_{n-3 DPA} | 7R, 14S-dihydroxy-8E, 10E, 12Z, 16Z, 19Z- |
| 1_{n-3 DPA} | | docosapentaenoic acid |
| Maresin | MaR2_{n-3 DPA} | 13R, 14S-dihydroxy-4Z, 7Z, 9E, 11E, 16Z, 19Z- |
| 2_{n-3 DPA} | | docosapentaenoic acid |
| 7S, 14S- | 7S, 14S- | 7S, 14Sdihydroxy- |
| diHDPA | diHDPA | 8E, 10Z, 12E, 16Z, 19Zdocosapentaenoic acid |
| 17-HDHA | | 17-hydroxy-docosa-7Z, 10Z, 13Z, 15E, 19Z-pentaenoic |
| | | acid |
| 14-HDHA | | 14S-hydroxy-docosa-7Z, 10Z, 12E, 16Z, 19Z-pentaenoic |
| | | acid |
| 13-HDHA | | 13-hydroxy-docosa-7Z, 10Z, 14E, 16Z, 19Z-pentaenoic |
| | | acid |
| 7-HDHA | | 7-hydroxy-docosa-8E, 10Z, 13Z, 16Z, 19Z-pentaenoic |
| | | acid |

### AA Metabolites

AA metabolites include Lipoxins—LXA₄, LXB₄, 5S, 15S-diHETE, 15R-LXA₄ and 15R-LXB₄ Leukotrienes: LTB₄, 5S, 12S-diHETE, 12-epi-LTB₄, 6-trans, 12-epi-LTB₄ and 20-OH-LTB₄, LTC₄, LTD₄ and LTE₄ and Prostanoids: PGD₂, PGE₂ and PGF₂ₐ TxB₂

Example AA metabolites are listed in Table 3 below:

TABLE 3

| Trivial name | Abbreviation | Full stereochemistry |
|---|---|---|
| Lipoxin A₄ | LXA4 | 5S, 6R, 15S-trihydroxy-7E, 9E, 11Z, 13E- |
| | | eicosatetraenoic acid |
| Lipoxin B₄ | LXB4 | 5S, 14R, 15S-trihydroxy-6E, 8Z, 10E, 12E- |
| | | eicosatetraenoic acid |
| 15R-Lipoxin A₄ | 15R-LXA₄ (15- | 5S, 6R, 15R-trihydroxy-7E, 9E, 11Z, 13E- |
| (15-epi-Lipoxin | epi-LXA₄) | eicosatetraenoic acid |
| A₄) | | |
| 15R-Lipoxin B₄ | 15R-LXB₄ (15- | 5S, 14R, 15R-trihydroxy-6E, 8Z, 10E, 12E- |
| (15-epi-Lipoxin | epi-LXB₄) | eicosatetraenoic acid |
| B₄) | | |
| 13, 14-dihydro, | 13, 14-dihydro, | 5S, 6R-dihydroxy-15-oxo-7E, 9E, 11Z- |
| 15-oxo-Lipoxin | 15-oxo-LXA₄ | eicosatrienoic acid |
| A₄ | | |
| 15-oxo-lipoxin | 15-oxo-LXA₄ | 5S, 6R-dihydroxy-15-oxo-7E, 9E, 11Z, 13E- |
| A₄ | | eicosatetraenoic acid |
| 5S, 15S- | 5S, 15S-diHETE | 5S, 15S-dihydroxy-6E, 8Z, 11Z, 13E- |
| dihydroxy- | | eicosatetraenoic acid |
| eicosatetraenoic | | |
| acid | | |
| Leukotriene B₄ | LTB₄ | 5S, 12R-dihydroxy-6Z, 8E, 10E, 14Z- |
| | | eicosatetraenoic acid |
| 5S, 12S-diHETE | 5S, 12S-diHETE | 5S, 12S-dihydroxy-6E, 8Z, 10E, 14Z- |
| | | eicosatetraenoic acid |
| Δ6-trans- | Δ6-trans-LTB₄ | 5S, 12R-dihydroxy-6E, 8E, 10E, 14Z- |
| leukotriene B⁴ | | eicosatetraenoic acid |
| Δ6-trans-12-epi- | Δ-6-trans, 12- | 5S, 12S-dihydroxy-6E, 8E, 10E, 14Z- |
| leukotriene B₄ | epi-LTB₄ | eicosatetraenoic acid |
| 20-hydroxy- | 20-OH-LTB₄ | 5S, 12R, 20-trihydroxy-6Z, 8E, 10E, 14Z- |
| Leukotriene B₄ | | eicosatetraenoic acid |
| Leukotriene C₄ | LTC₄ | 5S-hydroxy-6R-(S-glutathionyl)-7E, 9E, 11Z, 14Z- |
| | | eicosatetraenoic acid |
| Leukotriene D₄ | LTD₄ | 5S-hydroxy-6R-(S-cysteinylglycinyl)- |
| | | 7E, 9E, 11Z, 14Z-eicosatetraenoic acid |
| Leukotriene E₄ | LTE₄ | 5S-hydroxy-6R-(S-cysteinyl)-7E, 9E, 11Z, 14Z- |
| | | eicosatetraenoic acid |
| Prostaglandin | PGD₂ | 11-oxo-9a, 15S-dihydroxy-5Z, 13E-prostadienoic |
| D₂ | | acid |
| Prostaglandin | PGE₂ | 9-oxo-11α, 15S-dihydroxy-5Z, 13E-prostadienoic |
| E₂ | | acid |
| Prostaglandin | PGF₂ₐ | 9α, 11α, 15S-trihydroxy-5Z, 13E-prostadienoic |
| F_{2α} | | acid |
| Thromboxane | TXB₂ | 9α, 11, 15 S-trihydroxy-5Z, 13E-thrombadienoic |
| B₂ | | acid |
| 15-HETE | | 15-hydroxy-eicosa-5Z, 8Z, 11Z, 13E-tetraenoic |
| | | acid |
| 12-HETE | | 12-hydroxy-eicosa-5Z, 8Z, 10E, 14-tetraenoic acid |
| 5-HETE | | 5-hydroxy-eicosa-6E, 8Z, 11Z, 14Z, 1-tetraenoicic |
| | | acid |

### DHA Metabolites

DHA metabolites include the “D-series resolvins—RvD1, RvD2, RvD3, RvD4, RvD5, RvD6, 17R-RvD1 and 17R-RvD3, Protectins—PD1, 10S, 17S-diHDHA, 17R-PD1 and 22-OH-PD1, PCTR1, PCTR2 and PCTR3 and Maresins—MaR1, 7S, 14S-diHDHA, MaR2, 4S, 14S-diHDHA 5 and 22-OH-MaR1, MCTR1, MCTR2 and MCTR3”.

Example DHA metabolites are listed in Table 4 below:

TABLE 4

| Abbreviation | Trivial name | Full stereochemistry |
|---|---|---|
| RvD1 | Resolvin D1 | 7S, 8R, 17 S-trihydroxy-docosa-4Z, 9E, 11E, 13Z, 15E, 19Z- |
| | | hexaenoic acid |
| RvD2 | Resolvin D2 | 7S, 16R, 17S-trihydroxy-docosa- |
| | | 4Z, 8E, 10Z, 12E, 14E, 19Z-hexaenoic acid |
| RvD3 | Resolvin D3 | 4S, 7R, 17 S-trihydroxy-docosa-5Z, 7E, 9E, 13Z, 15E, 19Z- |
| | | hexaenoic acid |
| RvD4 | Resolvin D4 | 4S, 5R, 17S-trihydroxy-docosa-6E, 8E, 10Z, 13Z, 15E, 19Z- |
| | | hexaenoic acid |
| RvD5 | Resolvin D5 | 7S, 17S-dihydroxy-docosa-4Z, 8E, 10Z, 13Z, 15E, 19Z- |
| | | hexaenoic acid |
| RvD6 | Resolvin D6 | 4S, 17S-dihydroxy-docosa-5E, 7Z, 10Z, 13Z, 15E, 19Z- |
| | | hexaenoic acid |
| MaR1 | Maresin 1 | 7R, 14S-dihydroxy-docosa-4Z, 8E, 10E, 12Z, 16Z, 19Z- |
| | | hexaenoic acid |
| 4S, 14S- | | 4S, 14S-dihydroxy-docosa-5Z, 7E, 10E, 12Z, 16E, 19E- |
| diHDHA | | hexaenoic acid |
| 7S, 14S- | | 7S, 14S-dihydroxy-docosa-4Z, 8E, 10E, 12Z, 16E, 19E- |
| diHDHA | | hexaenoic acid |
| 14-oxo-MaR1 | | 14-oxo-7R,-hydroxy-docosa-4Z, 8E, 10E, 12Z, 16Z, 19Z- |
| | | hexaenoic acid |
| MCTR1 | Maresin | 13R-glutathionyl, 14S-hydroxy- |
| | Conjugate in | 4Z, 7Z, 9E, 11E, 13R, 14S, 16Z, 19Z-docosahexaenoic acid |
| | Tissue | |
| | Regeneration | |
| | 1 | |
| MCTR2 | Maresin | 13R-cysteinylglycinyl, 14S-hydroxy- |
| | Conjugate in | 4Z, 7Z, 9E, 11E, 13R, 14S, 16Z, 19Z-docosahexaenoic acid |
| | Tissue | |
| | Regeneration | |
| | 2 | |
| MCTR3 | Maresin | 13R-cysteinyl, 14S-hydroxy- |
| | Conjugate in | 4Z, 7Z, 9E, 11E, 13R, 14S, 16Z, 19Z-docosahexaenoic acid |
| | Tissue | |
| | Regeneration | |
| | 3 | |
| PD1 | Protectin D1 | 10R, 17S-dihydroxy-docosa-4Z, 7Z, 11E, 13E, 15Z, 19Z- |
| | | hexaenoic acid |
| 10S, 17S- | Protectin Dx | 10S, 17 S-dihydroxy-docosa-4Z, 7Z, 11E, 13Z, 15E, 19Z- |
| diHDHA | | hexaenoic acid |
| 17R-PD1 | 17R- | 10R, 17R-dihydroxy-docosa-4Z, 7Z, 11E, 13E, 15Z, 19Z- |
| | Protectin D1 | hexaenoic acid |
| 22-OH-PD1 | 22-OH- | 10R, 17S, 20-trihydroxy-docosa- |
| | Protectin D1 | 4Z, 7Z, 11E, 13E, 15Z, 19Z-hexaenoic acid |
| PCTR1 | Protectin | 16R-glutathionyl, 17S-hydroxy-4Z, 7Z, 10Z, 12E, 14E, 19Z- |
| | Conjugate in | docosahexaenoic acid |
| | Tissue | |
| | Regeneration | |
| | 1 | |
| PCTR2 | Protectin | 16R-cysteinylglycinyl, 17S-hydroxy- |
| | Conjugate in | 4Z, 7Z, 10Z, 12E, 14E, 19Z-docosahexaenoic acid |
| | Tissue | |
| | Regeneration | |
| | 2 | |
| PCTR3 | Protectin | 16R-cysteinyl, 17S-hydroxy-4Z, 7Z, 10Z, 12E, 14E, 19Z- |
| | Conjugate in | docosahexaenoic acid |
| | Tissue | |
| | Regeneration | |
| | 3 | |
| 17-HDHA | | 17-hydroxy-docosa-4Z, 7Z, 10Z, 13Z, 15E, 19Z-hexaenoic |
| | | acid |
| 14-HDHA | | 14S-hydroxy-docosa-4Z, 7Z, 10Z, 12E, 16Z, 19Z- |
| | | hexaenoic acid |
| 13-HDHA | | 13-hydroxy-docosa-4Z, 7Z, 10Z, 14E, 16Z, 19Z-hexaenoic |
| | | acid |
| 7-HDHA | | 7-hydroxy-docosa-4Z, 8E, 10Z, 13Z, 16Z, 19Z-hexaenoic |
| | | acid |
| 4-HDHA | | 4-hydroxy-docosa-5E, 7Z, 10Z, 13Z, 16Z, 19Z-hexaenoic |
| | | acid |

In some embodiments, the SPM is a DHA metabolite. In some embodiments, the SPM is an EPA metabolite.

In some embodiments, the SPM is selected from the group consisting of a maresin, a protectin, a resolvin and an E-series resolvin. In some embodiments, the SPM is a maresin.

In preferred embodiments, the SPM is a maresin conjugate in tissue regeneration (MCTR).

In some embodiments, the SPM is selected from the group consisting of MCTR3, MCTR1 and MCTR2.

In preferred embodiments, the SPM is MCTR3. In some embodiments, the SPM is MCTR2. In some embodiments, the SPM is MCTR1.

### Salts

The agents of the invention (e.g. the SPM or derivative thereof) can be present as salts, in particular pharmaceutically-acceptable salts or esters.

Pharmaceutically-acceptable salts of the agents of the invention include suitable acid addition or base salts thereof. A review of suitable pharmaceutical salts may be found in Berge. et al. (1977) J. Pharm. Sci. 66: 1-19. Salts are formed, for example, with strong inorganic acids such as mineral acids, e.g. sulphuric acid, phosphoric acid or hydrohalic acids; with strong organic carboxylic acids, e.g. alkanecarboxylic acids of 1 to 4 carbon atoms which are unsubstituted or substituted (e.g. by halogen), such as acetic acid; with saturated or unsaturated dicarboxylic acids, e.g. oxalic, malonic, succinic, maleic, fumaric, phthalic or tetraphthalic acid; with hydroxycarboxylic acids, e.g. ascorbic, glycolic, lactic, malic, tartaric or citric acid; with amino acids, e.g. aspartic or glutamic acid; with benzoic acid; or with organic sulfonic acids, such as (C₁-C₄)-alkyl- or aryl-sulfonic acids, which are unsubstituted or substituted (e.g. by a halogen), such as methane- or p-toluene sulfonic acid.

### Enantiomers/Tautomers

The invention also includes where appropriate all enantiomers and tautomers of the agent. The corresponding enantiomers and/or tautomers may be isolated/prepared by methods known in the art.

### Stereo and Geometric Isomers

Some of the agents of the invention may exist as stereoisomers and/or geometric isomers. For example, they may possess one or more asymmetric and/or geometric centres and so may exist in two or more stereoisomeric and/or geometric forms. The invention contemplates the use of all the individual stereoisomers and geometric isomers of those agents, and mixtures thereof. The terms used in the claims encompass these forms, provided said forms retain the appropriate functional activity (though not necessarily to the same degree).

The invention also includes all suitable isotopic variations of the agent or pharmaceutically-acceptable salts thereof. An isotopic variation of an agent of the invention or a pharmaceutically-acceptable salt thereof is defined as one in which at least one atom is replaced by an atom having the same atomic number, but an atomic mass different from the atomic mass usually found in nature. Examples of isotopes that can be incorporated into the agent and pharmaceutically-acceptable salts thereof include isotopes of hydrogen, carbon, nitrogen, oxygen, phosphorus, sulphur, fluorine and chlorine, such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically-acceptable salts thereof, for example, those in which a radioactive isotope such as ³H or ¹⁴C is incorporated, are useful in drug and/or substrate tissue distribution studies. Tritiated, i.e. ³H, and carbon-14, i.e. ¹⁴C, isotopes are particularly preferred for their ease of preparation and detectability. Further, substitution with isotopes such as deuterium, i.e. ²H, may afford certain therapeutic advantages resulting from greater metabolic stability, for example, increased in vivo half-life or reduced dosage requirements and hence may be preferred in some circumstances. Isotopic variations of the agent of the invention and pharmaceutically-acceptable salts thereof of this invention can generally be prepared by conventional procedures using appropriate isotopic variations of suitable reagents.

### Solvates

The invention also includes solvate forms of the agents of the invention. The terms used in the claims encompass these forms.

### Polymorphs

The invention also relates to the agents of the invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

### Cell

In another aspect, the invention provides a cell for use in a method of treating or preventing an inflammatory condition or disease.

In another aspect, the invention provides a cell for use in a method of treating or preventing rheumatoid arthritis (RA).

Suitably, the cell has been contacted with a specialized pro-resolving mediator (SPM) or derivative thereof. The cell may be, for example, a monocyte or a cell differentiated therefrom.

In another aspect, the invention provides a cell for use in a method of treating or preventing an inflammatory condition or disease, wherein the method comprises the step of contacting a monocyte with a specialized pro-resolving mediator (SPM) or derivative thereof to obtain the cell.

In another aspect, the invention provides a cell for use in a method of treating or preventing an inflammatory condition or disease, wherein the cell has been contacted with a specialized pro-resolving mediator (SPM) or derivative thereof.

In another aspect, the invention provides a cell for use in a method of treating or preventing an inflammatory condition or disease, wherein the method comprises the step of contacting a monocyte-derived macrophage (MDM) with a specialized pro-resolving mediator (SPM) or derivative thereof to obtain the cell.

In some embodiments, the inflammatory condition or disease is rheumatoid arthritis (RA), osteoarthritis or atherosclerosis.

In preferred embodiments, the inflammatory condition or disease is RA.

In another aspect, the invention provides a cell for use in a method of treating or preventing rheumatoid arthritis (RA), wherein the method comprises the step of contacting a monocyte with a specialized pro-resolving mediator (SPM) or derivative thereof to obtain the cell.

In another aspect, the invention provides a cell for use in a method of treating or preventing rheumatoid arthritis (RA), wherein the cell has been contacted with a specialized pro-resolving mediator (SPM) or derivative thereof.

In another aspect, the invention provides a cell for use in a method of treating or preventing rheumatoid arthritis (RA), wherein the method comprises the step of contacting a monocyte-derived macrophage (MDM) with a specialized pro-resolving mediator (SPM) or derivative thereof to obtain the cell.

Suitably, the cell may be part of a population of cells.

In some embodiments, the cell is a monocyte or a cell differentiated therefrom. In some embodiments, the cell is a monocyte-derived macrophage (MDM).

Monocytes are a type of leukocyte that may be capable of differentiating into macrophages and myeloid lineage dendritic cells. Monocytes may influence the process of adaptive immunity as a part of the vertebrate innate immune system.

Monocytes may comprise the CD14 marker (denoted as CD14+). Cluster of differentiation 14 (CD14) has been described as a monocyte/macrophage differentiation antigen on the surface of myeloid lineage and has been commonly used in normal tissue or blood as a marker for myeloid cells. In some embodiments, the monocyte is a CD14+ monocyte.

Three subclasses of monocytes have been classified in human blood, which may be identified based on their phenotypic receptors. Classical monocytes may be characterized by high level expression of the CD14 cell surface receptor (e.g. may be characterised as CD14highCD16low monocytes). Non-classical monocytes may be characterized as having low expression of CD14 and additional co-expression of the CD16 receptor (e.g. may be characterised as CD14lowCD16low monocytes). Intermediate monocytes may be characterized as having high level expression of CD14 and expression of CD16 (e.g. may be characterised as CD14highCD16high monocytes).

Suitably, the monocyte may be a classical monocyte, a non-classical monocyte or an intermediate monocyte.

Monocytes may be obtained from a tissue sample, e.g. a blood sample or a bone marrow sample. For example, monocytes may be obtained from peripheral blood (e.g. adult and foetal peripheral blood). Suitably, monocytes may be isolated from peripheral blood mononuclear cells (PBMCs).

Suitably, monocytes may be enriched.

In some embodiments, the cell is a monocyte-derived macrophage (MDM).

Macrophages are specialised white blood cells involved in the detection, phagocytosis and destruction of bacteria and other harmful organisms. They may also present antigens to T cells and initiate inflammation by releasing cytokines.

Macrophages are produced by the differentiation of monocytes in tissues. They may be identified their expression of proteins such as CD14, CD40, CD11b, CD64, EMR1, lysozyme M, MAC-1/MAC-3 and CD68.

Monocytes may be differentiated to MDMs by contacting (e.g. during culture) with one or more growth factor, for example M-CSF or GM-CSF.

In some embodiments, the population of monocytes is contacted with one or more growth factor. In some embodiments, the population of monocytes is contacted with M-CSF or GM-CSF. In some embodiments, the population of monocytes is contacted with serum, for example FBS. In some embodiments, the population of monocytes is contacted with the growth factor and the serum.

In some embodiments, the population of monocytes is contacted with the growth factor and/or serum for 1-7 days.

### Isolation and Enrichment of Cells

Suitably, the cell or population of cells is an isolated cell or population of cells.

The term “isolated” cell or population of cells as used herein may refer to the cell or population of cells having been previously removed from the body. An isolated cell or population of cells may be cultured and manipulated ex vivo or in vitro using standard techniques known in the art. An isolated cell or population of cells may later be reintroduced into a subject. Said subject may be the same subject from which the cells were originally isolated or a different subject.

A population of cells may be purified selectively for cells that exhibit a specific phenotype or characteristic, and from other cells which do not exhibit that phenotype or characteristic, or exhibit it to a lesser degree. For example, a population of cells that expresses a specific marker (such as CD14) may be purified from a starting population of cells. Alternatively, or in addition, a population of cells that does not express another marker (such as CD16) may be purified.

By “enriching” a population of cells for a certain type of cells it is to be understood that the concentration of that type of cells is increased within the population. The concentration of other types of cells may be concomitantly reduced.

Purification or enrichment may result in the population of cells being substantially pure of other types of cell.

Purifying or enriching for a population of cells expressing a specific marker (e.g. CD14) may be achieved by using an agent that binds to that marker, preferably substantially specifically to that marker.

An agent that binds to a cellular marker may be an antibody, for example an anti-CD14 antibody.

The term “antibody” refers to complete antibodies or antibody fragments capable of binding to a selected target, and including Fv, ScFv, F(ab′) and F(ab′)2, monoclonal and polyclonal antibodies, engineered antibodies including chimeric, CDR-grafted and humanised antibodies, and artificially selected antibodies produced using phage display or alternative techniques.

In addition, alternatives to classical antibodies may also be used in the invention, for example “avibodies”, “avimers”, “anticalins”, “nanobodies” and “DARPins”.

The agents that bind to specific markers may be labelled so as to be identifiable using any of a number of techniques known in the art. The agent may be inherently labelled, or may be modified by conjugating a label thereto. By “conjugating” it is to be understood that the agent and label are operably linked. This means that the agent and label are linked together in a manner which enables both to carry out their function (e.g. binding to a marker, allowing fluorescent identification or allowing separation when placed in a magnetic field) substantially unhindered. Suitable methods of conjugation are well known in the art and would be readily identifiable by the skilled person.

A label may allow, for example, the labelled agent and any cell to which it is bound to be purified from its environment (e.g. the agent may be labelled with a magnetic bead or an affinity tag, such as avidin), detected or both. Detectable markers suitable for use as a label include fluorophores (e.g. green, cherry, cyan and orange fluorescent proteins) and peptide tags (e.g. His tags, Myc tags, FLAG tags and HA tags).

A number of techniques for separating a population of cells expressing a specific marker are known in the art. These include magnetic bead-based separation technologies (e.g. closed-circuit magnetic bead-based separation), flow cytometry, fluorescence-activated cell sorting (FACS), affinity tag purification (e.g. using affinity columns or beads, such biotin columns to separate avidin-labelled agents) and microscopy-based techniques.

It may also be possible to perform the separation using a combination of different techniques, such as a magnetic bead-based separation step followed by sorting of the resulting population of cells for one or more additional (positive or negative) markers by flow cytometry.

### Method of Culture

In another aspect, the invention provides a method of culturing a population of monocytes, the method comprising:
(a) providing a population of monocytes; and
(b) contacting the population of monocytes with a specialized pro-resolving mediator (SPM) or derivative thereof.

In some embodiments, the monocytes are reprogrammed.

In another aspect, the invention provides a method of reprogramming monocytes, the method comprising:
(a) providing a population of monocytes; and
(b) contacting the population of monocytes with a specialized pro-resolving mediator (SPM) or derivative thereof.

The reprogramming of a monocyte through contact with the specialized pro-resolving mediator (SPM) or derivative thereof may provide a cell suitable for treating or preventing RA, preferably reducing joint inflammation, and/or repairing bone and/or cartilage.

Suitably, the monocytes may be differentiated during the method of the invention. The monocytes may be differentiated into monocyte-derived macrophages (MDMs). For example, the cells may be cultured under suitable conditions to differentiate, preferably to differentiate into MDMs.

In some embodiments, the population of monocytes is contacted with one or more growth factor, such as M-CSF or GM-CSF. In some embodiments, the population of monocytes is contacted with M-CSF or GM-CSF. In some embodiments, the population of monocytes is contacted with serum, for example FBS. In some embodiments, the population of monocytes is contacted with the growth factor and the serum.

In some embodiments, the population of monocytes is contacted with the growth factor and/or serum for 1-7 days, for example, 1-6, 1-5, 1-4, 1-3 or 1-2 days.

In another aspect, the invention provides a method of culturing a population of monocyte-derived macrophages (MDMs), the method comprising:
(a) providing a population of MDMs; and
(b) contacting the population of MDMs with a specialized pro-resolving mediator (SPM) or derivative thereof.

The cells may be cultured under suitable conditions (such as in a suitable medium and at a suitable temperature, and for example as disclosed in the Examples herein), which may be readily selected by the skilled person.

The cells may be cultured in a suitable medium, such as Dulbecco's Modified Eagle Medium (DMEM). The cells may be cultured at a suitable temperature, such as at 37° C., and/or in the presence of suitable CO₂ levels, such as 5% CO₂.

### Method of Treatment

In another aspect the invention provides a specialized pro-resolving mediator (SPM) or derivative thereof or cell of the invention for use in therapy, preferably for use in treating or preventing rheumatoid arthritis (RA).

In another aspect, the invention provides a method of treating or preventing an inflammatory condition or disease comprising administering a specialized pro-resolving mediator (SPM) or derivative thereof to a subject in need thereof.

In another aspect, the invention provides a method of treating or preventing an inflammatory condition or disease comprising the steps:
(a) providing a population of monocytes;
(b) contacting the population of monocytes with a specialized pro-resolving mediator (SPM) or derivative thereof; and
(c) administering the cells from step (b) or a portion thereof to a subject in need thereof.

In another aspect, the invention provides a method of treating or preventing an inflammatory condition or disease comprising the steps:
(a) providing a population of monocyte-derived macrophages (MDMs);
(b) contacting the population of MDMs with a specialized pro-resolving mediator (SPM) or derivative thereof; and
(c) administering the cells from step (b) or a portion thereof to a subject in need thereof.

In some embodiments, the inflammatory condition or disease is rheumatoid arthritis (RA), osteoarthritis or atherosclerosis.

In preferred embodiments, the inflammatory condition or disease is RA.

In another aspect, the invention provides a method of treating or preventing rheumatoid arthritis (RA) comprising administering a specialized pro-resolving mediator (SPM) or derivative thereof to a subject in need thereof.

In another aspect, the invention provides a method of treating or preventing rheumatoid arthritis (RA) comprising the steps:
(a) providing a population of monocytes;
(b) contacting the population of monocytes with a specialized pro-resolving mediator (SPM) or derivative thereof; and
(c) administering the cells from step (b) or a portion thereof to a subject in need thereof.

In another aspect, the invention provides a method of treating or preventing rheumatoid arthritis (RA) comprising the steps:
(a) providing a population of monocyte-derived macrophages (MDMs);
(b) contacting the population of MDMs with a specialized pro-resolving mediator (SPM) or derivative thereof; and
(c) administering the cells from step (b) or a portion thereof to a subject in need thereof.

In another aspect, the invention provides a method of reducing or preventing joint inflammation in a subject having an inflammatory condition or disease comprising administering a specialized pro-resolving mediator (SPM) or derivative thereof to a subject in need thereof.

In another aspect, the invention provides a method of reducing or preventing joint inflammation in a subject having an inflammatory condition or disease comprising the steps:
(a) providing a population of monocytes;
(b) contacting the population of monocytes with a specialized pro-resolving mediator (SPM) or derivative thereof; and
(c) administering the cells from step (b) or a portion thereof to a subject in need thereof.

In another aspect, the invention provides a method of reducing or preventing joint inflammation in a subject having an inflammatory condition or disease comprising the steps:
(a) providing a population of monocyte-derived macrophages (MDMs);
(b) contacting the population of MDMs with a specialized pro-resolving mediator (SPM) or derivative thereof; and
(c) administering the cells from step (b) or a portion thereof to a subject in need thereof.

In another aspect, the invention provides a method of repairing bone and/or cartilage in a subject having an inflammatory condition or disease comprising administering a specialized pro-resolving mediator (SPM) or derivative thereof to a subject in need thereof.

In another aspect, the invention provides a method of repairing bone and/or cartilage in a subject having an inflammatory condition or disease comprising the steps:
(a) providing a population of monocytes;
(b) contacting the population of monocytes with a specialized pro-resolving mediator (SPM) or derivative thereof; and
(c) administering the cells from step (b) or a portion thereof to a subject in need thereof.

In another aspect, the invention provides a method of repairing bone and/or cartilage in a subject having an inflammatory condition or disease comprising the steps:
(a) providing a population of monocyte-derived macrophages (MDMs);
(b) contacting the population of MDMs with a specialized pro-resolving mediator (SPM) or derivative thereof; and
(c) administering the cells from step (b) or a portion thereof to a subject in need thereof.

In some embodiments, the inflammatory condition or disease is rheumatoid arthritis (RA), osteoarthritis or atherosclerosis.

In preferred embodiments, the inflammatory condition or disease is RA.

In another aspect, the invention provides a method of reducing or preventing joint inflammation in a subject having rheumatoid arthritis (RA) comprising administering a specialized pro-resolving mediator (SPM) or derivative thereof to a subject in need thereof.

In another aspect, the invention provides a method of reducing or preventing joint inflammation in a subject having rheumatoid arthritis (RA) comprising the steps:
(a) providing a population of monocytes;
(b) contacting the population of monocytes with a specialized pro-resolving mediator (SPM) or derivative thereof; and
(c) administering the cells from step (b) or a portion thereof to a subject in need thereof.

In another aspect, the invention provides a method of reducing or preventing joint inflammation in a subject having rheumatoid arthritis (RA) comprising the steps:
(a) providing a population of monocyte-derived macrophages (MDMs);
(b) contacting the population of MDMs with a specialized pro-resolving mediator (SPM) or derivative thereof; and
(c) administering the cells from step (b) or a portion thereof to a subject in need thereof.

In another aspect, the invention provides a method of repairing bone and/or cartilage in a subject having rheumatoid arthritis (RA) comprising administering a specialized pro-resolving mediator (SPM) or derivative thereof to a subject in need thereof.

In another aspect, the invention provides a method of repairing bone and/or cartilage in a subject having rheumatoid arthritis (RA) comprising the steps:
(a) providing a population of monocytes;
(b) contacting the population of monocytes with a specialized pro-resolving mediator (SPM) or derivative thereof; and
(c) administering the cells from step (b) or a portion thereof to a subject in need thereof.

In another aspect, the invention provides a method of repairing bone and/or cartilage in a subject having rheumatoid arthritis (RA) comprising the steps:
(a) providing a population of monocyte-derived macrophages (MDMs);
(b) contacting the population of MDMs with a specialized pro-resolving mediator (SPM) or derivative thereof; and
(c) administering the cells from step (b) or a portion thereof to a subject in need thereof.

All references herein to treatment include curative, palliative and prophylactic treatment. The treatment of mammals, particularly humans, is preferred. Both human and veterinary treatments are within the scope of the invention.

In some embodiments, a cell or population of cells prepared according to a method of the invention is administered as part of an autologous transplant procedure.

In another embodiment, a cell or population of cells prepared according to a method of the invention is administered as part of an allogeneic transplant procedure.

The term “autologous transplant procedure” as used herein refers to a procedure in which the starting cells (which may then be cultured according to a method of the invention) is obtained from the same subject as that to which the cultured cells are administered. Autologous transplant procedures are advantageous as they avoid problems associated with immunological incompatibility and are available to subjects irrespective of the availability of a genetically matched donor.

The term “allogeneic transplant procedure” as used herein refers to a procedure in which the starting cells (which may then be cultured according to a method of the invention) is obtained from a different subject as that to which the cultured cells are administered. Preferably, the donor will be genetically matched to the subject to which the cells are administered to minimise the risk of immunological incompatibility.

### Response to Therapies in Patients

Methods of assessing a subject's response to a therapy, for example for rheumatoid arthritis, are known in the art and would be familiar to a skilled person.

By way of example, well known measures of disease activity in RA include the Disease Activity Score (DAS), a modified version DAS28, and the DAS-based EULAR response criteria.

The assessment of response to a therapy for rheumatoid arthritis may use the Clinical Disease Activity Index (CDAI).

Other measures of assessment of response to a therapy for rheumatoid arthritis include CDAI-remission, DAS28(ESR)/(CRP) moderate/good EULAR-response, DAS28(ESR)/(CRP) low-disease-activity, DAS28(ESR)/(CRP) remission and patient reported outcomes, such as fatigue.

In some embodiments, the treatment, for example of RA, reduces joint inflammation. In some embodiments, the treatment, for example of RA, reduces joint oedema.

Suitably, joint inflammation may be reduced by 10%, 20%, 30%, 40%, 50%, 60%, 70%, 80%, 90% or 100% compared to the joint inflammation before the treatment.

Joint inflammation may be measured using methods known in the art and familiar to the skilled person, such as using magnetic resonance imaging (MRI) and physical measurement of joint swelling.

In preferred embodiments, the treatment, for example of RA, repairs bone and/or cartilage. In preferred embodiments, the treatment, for example of RA, reduces bone and/or cartilage damage (e.g. increases bone and/or cartilage volume).

In preferred embodiments, the treatment, for example of RA, increases bone and/or cartilage volume. In some embodiments, the treatment, for example of RA, increases expression of collagen 2 and/or collagen X. In some embodiments, the treatment, for example of RA, increases bone and/or cartilage integrity.

Suitably, bone and/or cartilage damage may be reduced by 10%, 20%, 30%, 40%, 50%, 60%, 70%, 80%, 90% or 100% compared to the bone and/or cartilage volume before the onset of the disease or conditions, for example RA.

Bone damage and/or repair may be measured using methods known in the art and familiar to the skilled person (e.g. to determine bone volume), such as using magnetic resonance imaging (MRI) and computed tomography (CT) scans.

Cartilage damage and/or repair may be measured using methods known in the art and familiar to the skilled person (e.g. to determine cartilage volume), such as using ultrasound.

### Pharmaceutical Compositions and Injected Solutions

Although the agents for use in the invention can be administered alone, they will generally be administered in admixture with a pharmaceutical carrier, excipient and/or diluent, particularly for human therapy.

The medicaments, for example the SPM or derivative thereof or cell, of the invention may be formulated into pharmaceutical compositions. These compositions may comprise, in addition to the medicament, a pharmaceutically acceptable carrier, diluent, excipient, buffer, stabiliser or other materials well known in the art. Such materials should be non-toxic and should not interfere with the efficacy of the active ingredient. The precise nature of the carrier or other material may be determined by the skilled person according to the route of administration, e.g. intravenous or intra-articular.

The pharmaceutical composition is typically in liquid form. Liquid pharmaceutical compositions generally include a liquid carrier such as water, petroleum, animal or vegetable oils, mineral oil or synthetic oil. Physiological saline solution, magnesium chloride, dextrose or other saccharide solution, or glycols such as ethylene glycol, propylene glycol or polyethylene glycol may be included. In some cases, a surfactant, such as pluronic acid (PF68) 0.001% may be used. In some cases, serum albumin may be used in the composition.

For injection, the active ingredient may be in the form of an aqueous solution which is pyrogen-free, and has suitable pH, isotonicity and stability. The skilled person is well able to prepare suitable solutions using, for example, isotonic vehicles such as Sodium Chloride Injection,

Ringer's Injection or Lactated Ringer's Injection. Preservatives, stabilisers, buffers, antioxidants and/or other additives may be included as required.

For delayed release, the medicament may be included in a pharmaceutical composition which is formulated for slow release, such as in microcapsules formed from biocompatible polymers or in liposomal carrier systems according to methods known in the art.

Handling of the cell therapy products is preferably performed in compliance with FACT-JACIE International Standards for cellular therapy.

### Administration

In some embodiments, the SPM or derivative thereof or cell is administered to a subject systemically.

In some embodiments, the SPM or derivative thereof or cell is administered to a subject locally.

The term “systemic delivery” or “systemic administration” as used herein means that the agent of the invention is administered into the circulatory system, for example to achieve broad distribution of the agent. In contrast, topical or local administration restricts the delivery of the agent to a localised area.

In some embodiments, the SPM or derivative thereof or cell is administered intravascularly, intravenously or intra-arterially.

In some embodiments, the SPM or derivative thereof or cell is administered intravenously. In some embodiments, the SPM or derivative thereof or cell is administered intra-articularly.

### Dosage

The skilled person can readily determine an appropriate dose of an agent of the invention to administer to a subject. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. There can of course be individual instances where higher or lower dosage ranges are merited, and such are within the scope of the invention.

### Subject

The term “subject” as used herein refers to either a human or non-human animal.

Examples of non-human animals include vertebrates, for example mammals, such as non-human primates (particularly higher primates), dogs, rodents (e.g. mice, rats or guinea pigs), pigs and cats. The non-human animal may be a companion animal.

Preferably, the subject is human.

In some embodiments the subject is an adult human. In some embodiments, the subject is a child or an infant.

In some embodiments, the subject is suspected of having an inflammatory condition or disease, for example rheumatoid arthritis (RA), osteoarthritis or atherosclerosis. In some embodiments, the subject is suspected of having RA.

In some embodiments, the SPM or derivative thereof or cell is administered to a subject after onset of the inflammatory condition or disease, for example rheumatoid arthritis (RA), osteoarthritis or atherosclerosis. In some embodiments, the SPM or derivative thereof or cell is administered to a subject after RA onset.

In some embodiments, the subject presents one or more symptoms associated with the inflammatory condition or disease, for example rheumatoid arthritis (RA), osteoarthritis or atherosclerosis. In some embodiments, the subject has been diagnosed with the inflammatory condition or disease, for example rheumatoid arthritis (RA), osteoarthritis or atherosclerosis.

In some embodiments, the subject presents one or more symptoms associated with RA. In some embodiments, the subject has been diagnosed with RA.

In some embodiments, the subject has presented one or more symptoms of the inflammatory condition or disease, for example rheumatoid arthritis (RA), osteoarthritis or atherosclerosis for less than 1 year, for example less than 11, 10, 9, 8, 7, 6, 5, 4 or 3 months.

In some embodiments, the subject has presented one or more symptoms of rheumatoid arthritis for less than 1 year, for example less than 11, 10, 9, 8, 7, 6, 5, 4 or 3 months.

In some embodiments, the subject has failed DMARD treatment.

### Method of Diagnosis

In another aspect, the invention provides a method of diagnosing an inflammatory condition or disease comprising the steps:
(a) determining the level of one or more maresin conjugate in tissue regeneration (MCTR) in one or more sample obtained from a subject;
(b) comparing the level of the one or more MCTR to one or more corresponding reference value;
   wherein the level of the one or more MCTR compared to the one or more reference value is indicative of the inflammatory condition or disease.

In some embodiments, the inflammatory condition or disease is rheumatoid arthritis (RA), osteoarthritis or atherosclerosis.

In preferred embodiments, the inflammatory condition or disease is RA.

In another aspect, the invention provides a method of diagnosing rheumatoid arthritis (RA) comprising the steps:
(a) determining the level of one or more maresin conjugate in tissue regeneration (MCTR) in one or more sample obtained from a subject;
(b) comparing the level of the one or more MCTR to one or more corresponding reference value;
   wherein the level of the one or more MCTR compared to the one or more reference value is indicative of RA.

In some embodiments, the sample is a plasma or whole blood sample. In some embodiments, the sample is a plasma sample.

In some embodiments, the MCTR is selected from the group consisting of MCTR3, MCTR1 and MCTR2. In preferred embodiments, the MCTR is MCTR3.

Suitably, the subject may be a subject suspected of having the inflammatory condition or disease, for example rheumatoid arthritis (RA), osteoarthritis or atherosclerosis.

Suitably, the subject may be a subject suspected of having RA.

Methods for obtaining samples are well known in the art and would be familiar to the skilled person.

### Determining MCTR Level

The level of the one or more MCTR may be determined using any suitable method known in the art, for example as disclosed in the Examples herein.

Suitably, the level of the one or more MCTR may be determined using liquid chromatography tandem mass spectrometry (LC-MS/MS) after extracting the one or more MCTR from the sample(s). MCTRs may be extracted from samples using solid-phase extraction, for instance using C18 columns.

One or more internal labelled standard, e.g. deuterium-labelled standard, may be added to the sample(s) prior to extraction of the one or more MCTR to facilitate quantitation of the one or more MCTR.

The level of the one or more MCTR may be determined using a homogeneous or heterogeneous immunoassay.

Suitably, the immunoassay may comprise an enzyme immunoassay (EIA) in which the label is an enzyme such, for example, as horseradish peroxidase (HRP). Suitable substrates for HRP are well known in the art and include, for example, ABTS, OPD, AmplexRed, DAB, AEC, TMB, homovanillic acid and luminol. In some embodiments, an ELISA immunoassay may be used; a sandwich ELISA assay may be particularly preferred. The immunoassay may be, for example, competitive or non-competitive.

Measuring MCTR levels may be by equipment for measuring the level of a specific MCTR in a sample comprising a sample collection device and an immunoassay. The equipment may further comprise a detector for detecting labelled MCTR or labelled antibodies to the MCTR in the immunoassay. In preferred embodiments, the label may be an enzyme having a chromogenic or chemiluminescent substrate that is coloured or caused or allowed to fluoresce when acted on by the enzyme. The immunoassay or equipment may be incorporated into a miniaturised device for measuring the level of at least one MCTR in a biological sample. Suitably, the device may comprise a lab-on-a-chip.

### Reference Values

The method of the invention comprises the step of comparing the level of one or more biomarker to one or more corresponding reference values.

As used herein, the term “reference value” may refer to a level against which another level (e.g. the level of one or more biomarker disclosed herein) is compared (e.g. to make a diagnostic (e.g. predictive and/or prognostic) and/or therapeutic determination).

For example, the reference value may be derived from levels in a reference population (e.g. the median level in a reference population), for example a population of patients having RA; a reference sample; and/or a pre-assigned value (e.g. a cut-off value which was previously determined to significantly separate a first subset of individuals who had rheumatoid arthritis and a second subset of individuals who did not).

In some embodiments, the cut-off value may be the median or mean level in the reference population. In some embodiments, the reference level may be the top 40%, the top 30%, the top 20%, the top 10%, the top 5% or the top 1% of the level in the reference population.

A corresponding reference value may be derived from a subject without RA, for example a subject with osteoarthritis (OA).

The reference value may, for example, be based on a mean or median level of the biomarker in a control population of subjects, e.g. 5, 10, 100, 1000 or more subjects (who may be age- and/or gender-matched, or unmatched to the test subject).

In certain embodiments the reference value may have been previously determined, or may be calculated or extrapolated without having to perform a corresponding determination on a control sample with respect to each test sample obtained.

The skilled person will understand that they can combine all features of the invention disclosed herein without departing from the scope of the invention as disclosed.

Preferred features and embodiments of the invention will now be described by way of non-limiting examples.

The practice of the present invention will employ, unless otherwise indicated, conventional techniques of chemistry, biochemistry, molecular biology, microbiology and immunology, which are within the capabilities of a person of ordinary skill in the art. Such techniques are explained in the literature. See, for example, Sambrook, J., Fritsch, E. F. and Maniatis, T. (1989) Molecular Cloning: A Laboratory Manual, 2nd Edition, Cold Spring Harbor Laboratory Press; Ausubel, F. M. et al. (1995 and periodic supplements) Current Protocols in Molecular Biology, Ch. 9, 13 and 16, John Wiley & Sons; Roe, B., Crabtree, J. and Kahn, A. (1996) DNA Isolation and Sequencing: Essential Techniques, John Wiley & Sons; Polak, J. M. and McGee, J.O'D. (1990) In Situ Hybridization: Principles and Practice, Oxford University Press; Gait, M. J. (1984) Oligonucleotide Synthesis: A Practical Approach, IRL Press; and Lilley, D. M. and Dahlberg, J. E. (1992) Methods in Enzymology: DNA Structures Part A: Synthesis and Physical Analysis of DNA, Academic Press. Each of these general texts is herein incorporated by reference.

### EXAMPLES

### Example 1

### Results

MCTR3 Negatively Correlates with Joint Disease Activity in Humans

Circulating lipid mediator concentrations are linked with peripheral organ disease activity, since these autacoids influence leukocyte recruitment and activation status. To establish whether there was a link between disease activity and MCTR concentrations in RA patients we investigated plasma levels of these molecules in relation to both systemic and joint disease activity markers. Plasma was obtained from The Pathobiology of Early Arthritis Cohort (PEAC), which is a highly phenotyped patient cohort of disease-modifying anti-rheumatic drugs (DMARD)-naïve patients. Using lipid mediator profiling, we identified all three mediators in plasma from these patients. Concentrations of all three MCTRs were observed to display a negative correlation with joint disease activity (i.e. DAS28 scores), plasma C-reactive protein and erythrocyte sedimentation rate (Table 5).

TABLE 5
Correlation between peripheral blood MCTR3 concentrations and disease
activity in DMARD naive RA patients

| | MCTR1 | MCTR2 | MCTR3 |
|---|---|---|---|
| ESR | r = 0.06797; (CI = −0.14 | r = −0.1464; (CI = −0.34 | r = −0.2851; (CI = −0.46 |
| | to 0.27); p = 0.506 | to 0.06); p = 0.150 | to −0.09) p = 0.004 |
| CRP | r = 0.03728; (CI = −0.17 | r = −0.2041; (CI = −0.39 | r = −0.2471; (CI = −0.43 |
| | to 0.24); p = 0.718 | to 0.002); p = 0.046 | to −0.04); p = 0.015 |
| DAS28 | r = −0.08457; (CI = | r = −0.1730; (CI = −0.36 | r = −0.3676; (CI = −0.53 |
| | −0.28 to 0.12); p = 0.405 | to 0.03); p = 0.087 | to −0.18); p = 0.0002 |

Plasma was collected from a patient cohort of DMARD naive patients (n=99 patients) and plasma concentrations for MCTR1, MCTR2 and MCTR3 were established using lipid mediator profiling (see methods for details). Concentrations for each of these meditators were then correlated with DAS28 scores as well as plasma C-reactive protein (CRP) and erythrocyte sedimentation rate (ESR) using Spearman correlation.

### MCTR3 Displays Anti-Arthritic Activity in Inflammatory Arthritis

Having observed a significant relationship between MCTR3 concentrations and disease activity we next questioned whether MCTR3 displayed joint protective actions. For this purpose, we employed a serum transfer model of inflammatory arthritis, which relies on the activation of the innate immune system replicating the effector phase of rheumatoid arthritis (Korganow et al., 1999, Immunity 10, 451-461). MCTR3 administration immediately after disease onset conferred protection against joint inflammation as observed by a significant reduction in clinical scores, and improvements in histological markers of disease, such as a decrease in leukocyte infiltration, and increased safranin staining, a measure of glycosaminoglycan content in the cartilage (FIG. 7A, B, C). We next evaluated whether MCTR3 administration also regulated the joint lipid mediator profile. Using partial least square discriminant analysis, which produces a regression model built using concentrations of lipid mediators differently expressed between the two groups, we found a shift in joint lipid mediator concentrations in mice treated with MCTR3. This shift was linked with a downregulation of pro-inflammatory and nociceptive eicosanoids including PGE₂ and PGF₂ₐ and an upregulation of pro-resolving and anti-nociceptive mediators such as MaR1 and PDX (FIG. 7D,E).

We next tested whether MCTR3 also displayed joint protective activities when administrated later in the disease course. For this purpose, we used a model of sustained joint inflammation (Archer et al., 2016, J Transl Med 14, 170). MCTR3 was administered 10 days after disease onset and joint inflammation was evaluated throughout the disease course. Here we found that treatment of mice with MCTR3 accelerated the resolution of joint inflammation as demonstrated by a shortening of the resolution interval form ˜9 days to ˜5 days, a significant reduction in clinical scores and a marked reduction in joint oedema (FIG. 1A,B). Histological evaluation of joints collected from these mice demonstrated that MCTR3 reduced all the parameters evaluated, significantly reducing both leukocyte infiltration and cartilage damage (FIG. 8).

Mononuclear phagocytes, in particular MDM, play a central role in the propagation and termination of inflammation as well as tissue repair and regeneration. Therefore, we next evaluated whether MCTR3 governed MDM phenotype in arthritic joints. Flow-cytometric evaluation of phenotypic markers in cells isolated from mice treated with MCTR3 demonstrated a marked shift in phenotype as highlighted by a shift in the cluster representing cells obtained from these mice when compared with cells isolated from mice treated with Vehicle alone (FIG. 1C, FIG. 9). To evaluate which of the phenotypic markers were responsible for this shift in macrophage markers we evaluated the Variable in Importance (VIP) scores, whereby a VIP score >1 identifies those variables that contribute to the observed separation between the two groups. This demonstrated that the shift in phenotype was primarily linked with the upregulation of two markers in cells from MCTR3 treated mice, namely Arginase (Arg)-1 and Interleukin (IL)-10 (FIG. 1D).

We next assessed whether the protective activities of MCTR3 were retained in a model of adaptive immune system driven arthritis, using the glucose-6-phosphate isomerase peptide driven model of inflammatory arthritis (Schubert et al., 2004, J Immunol 172, 4503-4509). Here, administration of MCTR3 during the course of arthritic inflammation also led to a reduction in joint inflammation as measured by a reduction in both clinical scores and joint oedema (FIG. 1E,F). Notably, the MDM activities of MCTR3 were also retained in this model, as demonstrated by the marked shift in the macrophage phenotype observed in cells isolated from joints of MCTR3 treated mice when compared with those isolated from joints of vehicle treated mice (FIG. 1G). This shift in phenotype was linked with an upregulation of three phenotypic markers, including Arg-1 and CD11b, in cells obtained from joints of MCTR3 treated mice (FIG. 1H). Taken together these findings suggest that MCTR3 treatment alters joint MDM phenotype and reduces arthritic inflammation.

### MCTR3 Promotes Bone and Cartilage Repair

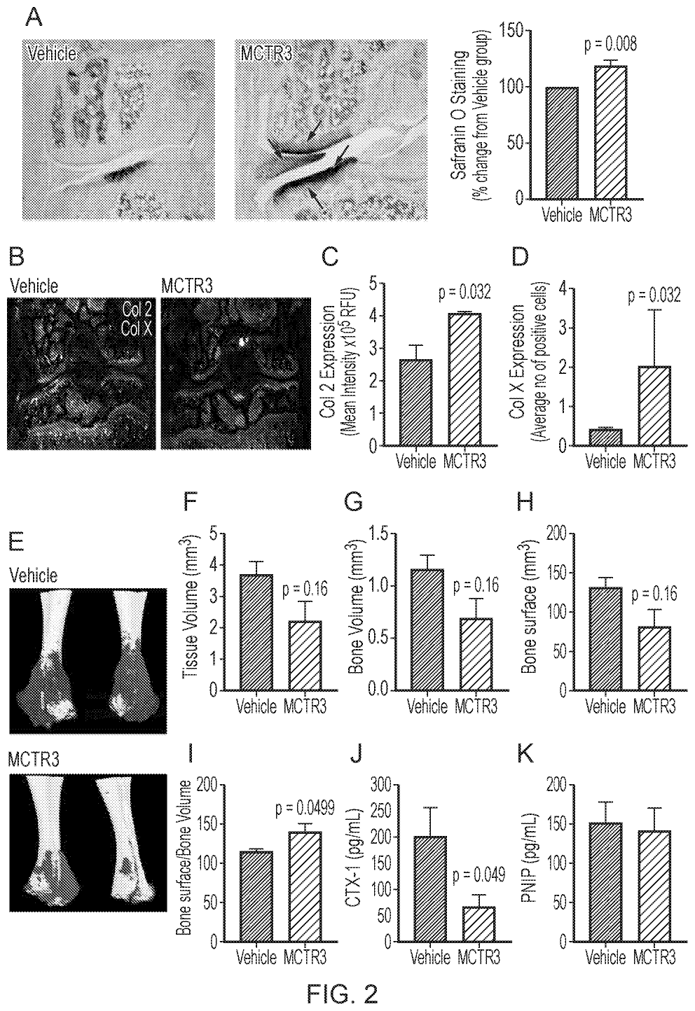

Chronic inflammation in RA is associated with both cartilage and bone degradation which is the main cause of debilitation in patients with RA. Thus, we next questioned whether MCTR3, in addition to reducing joint inflammation and cartilage damage also promoted joint repair. To address this question, we investigated whether MCTR3 regulated cartilage repair in arthritic mice. For this purpose, we used safranin O-staining to evaluate glycosaminoglycan content in joints from arthritic mice. Here we observed significantly higher safranin O-staining in joints from MCTR3 treated mice when compared with vehicle treated mice. Of note these cartilage protective activities of MCTR3 were observed in mice that were challenged with K/B×N serum as well as those challenged with glucose-6-phosphate isomerase peptide (FIG. 2A and FIG. 10A, B). Furthermore, immunohistochemical staining of joints from MCTR3 treated mice demonstrated an increase in the expression of both collagen 2, the principal molecular component in mammalian cartilage, and that of collagen X, which is expressed in the calcified zone of cartilage that interfaces with bone (FIG. 2B-D). Thus, these results demonstrate that MCTR3 displays cartilage-protective activities in inflammatory arthritis.

Since one of the main debilitating features of arthritis is bone erosion, we next determined whether the joint protective actions of MCTR3 also extended to the bone. For this purpose, we used microCT analysis to investigate bone volume in arthritic joints, comparing bone volumes on day 24, prior to MCTR3 treatment, to those at day 35. This analysis demonstrated that bone volume in vehicle treated arthritic mice was reduced, in line with the sustained disease activity (FIG. 1E,F and FIG. 10C), whereas bone volume in MCTR3 treated mice was increased (FIG. 9C). We next queried whether these bone protective activities of

MCTR3 were linked with an acceleration of bone repair. To test this, we assessed the bone callus size in arthritic mice treated with MCTR3. Analysis using microCT demonstrated smaller bone calluses on MCTR3 treated mice, as measured by the assessment of the total volume and surface area occupied by the callus (FIG. 2E-H). Notably, these changes were linked with a significant increase in overall surface to volume ratio in the callus a marker of callus mineralization and therefore bone integrity (FIG. 21). Assessment of plasma concentrations of C-terminal telopeptide (CTX), a marker of bone resorption, and amino-terminal propeptide of type I collagen (PINP), a marker of bone formation, demonstrated a significant reduction in plasma CTX concentrations in MCTR3 treated mice, whereas PINP levels were similar between the two groups (FIG. 2J, K). These findings indicate that MCTR3 reduces the increased bone and cartilage turnover, characteristic of arthritic inflammation thereby suggesting that this autacoid activates protective mechanisms to improve both cartilage and bone integrity in inflammatory arthritis.

### MCTR3 Reprograms Monocyte Responses to Reduce Inflammation and Repair Inflamed Joints

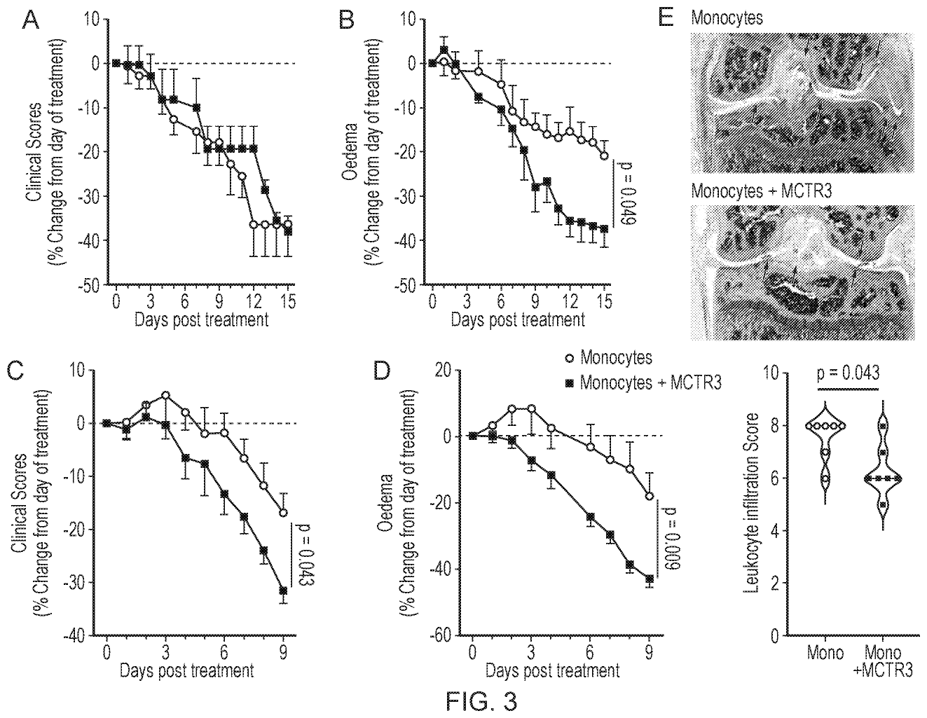

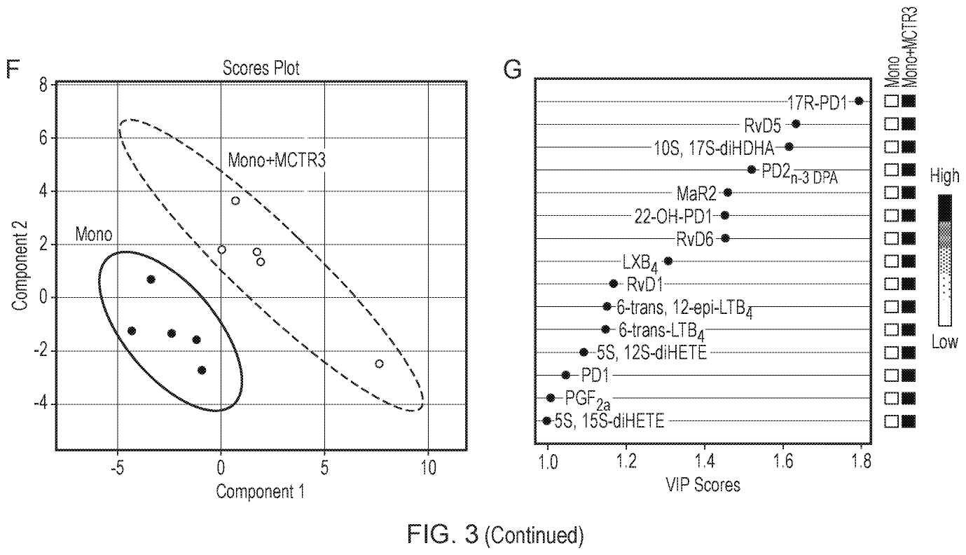

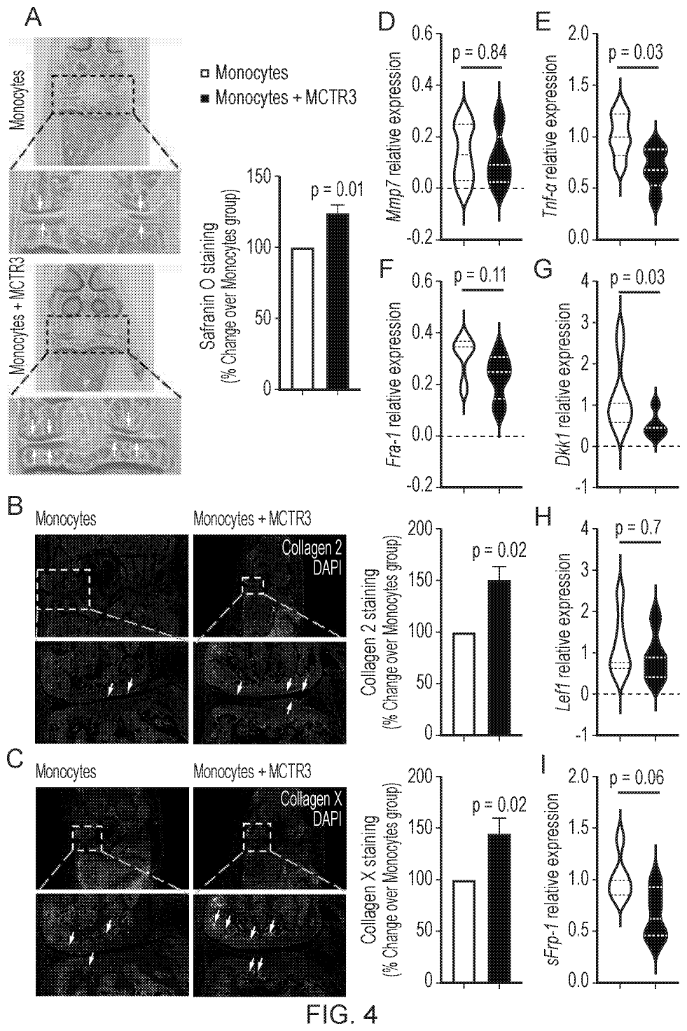

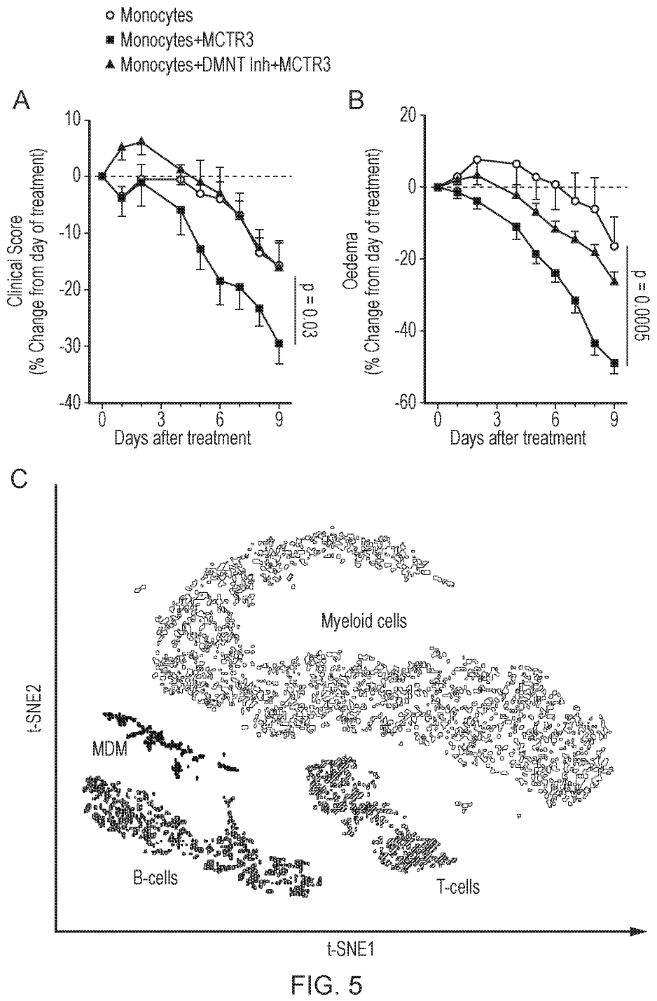

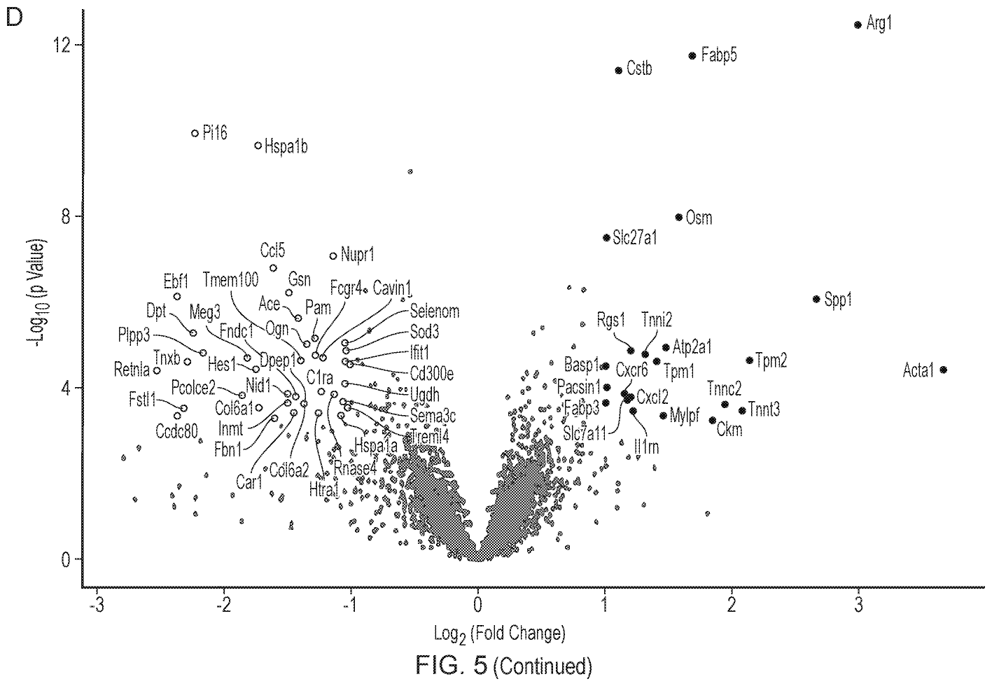

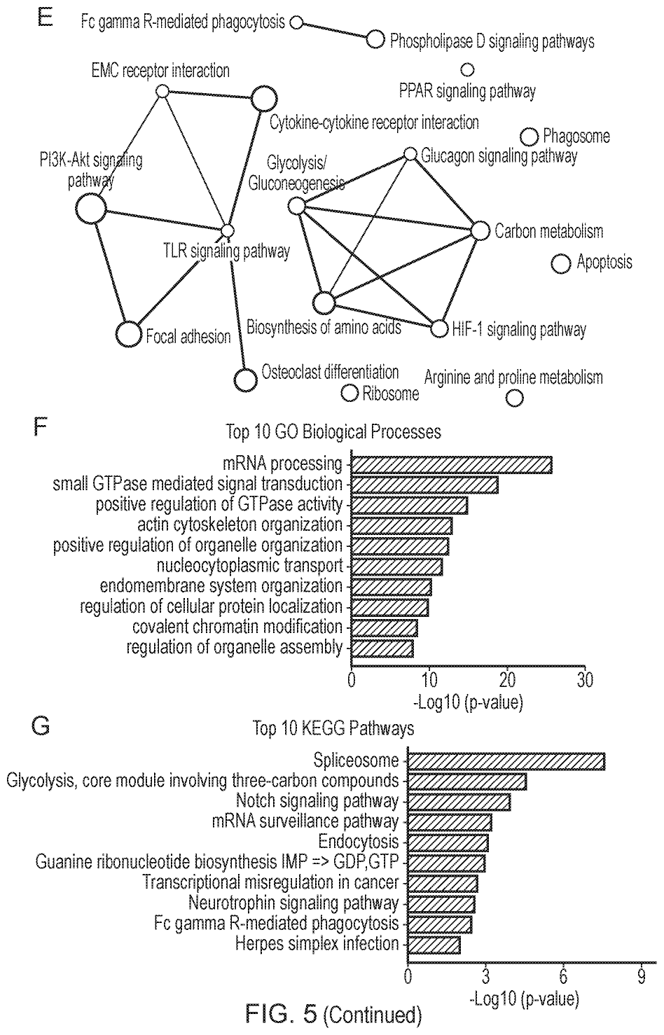

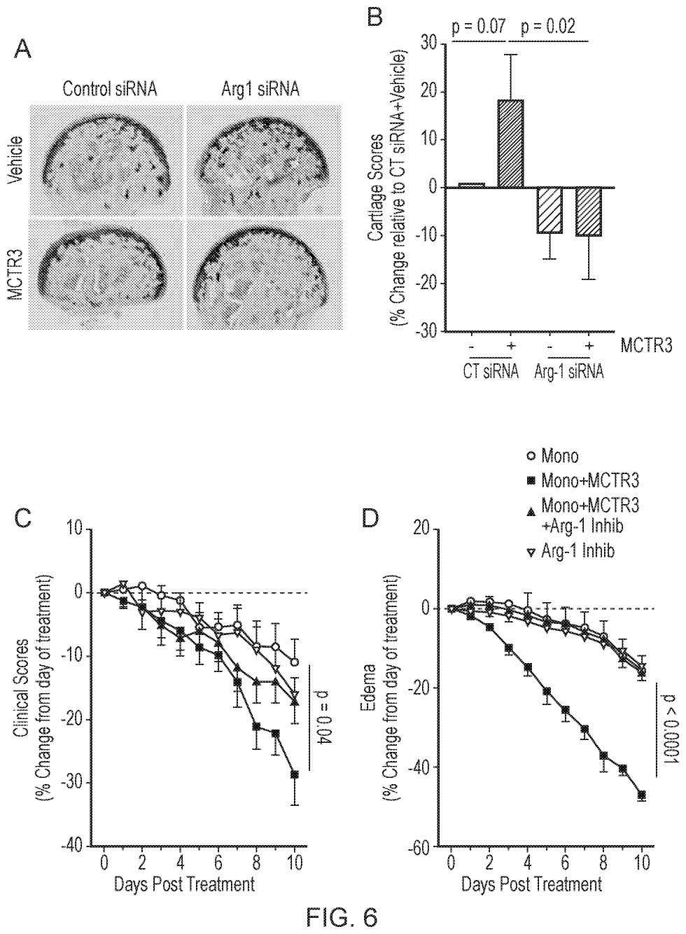

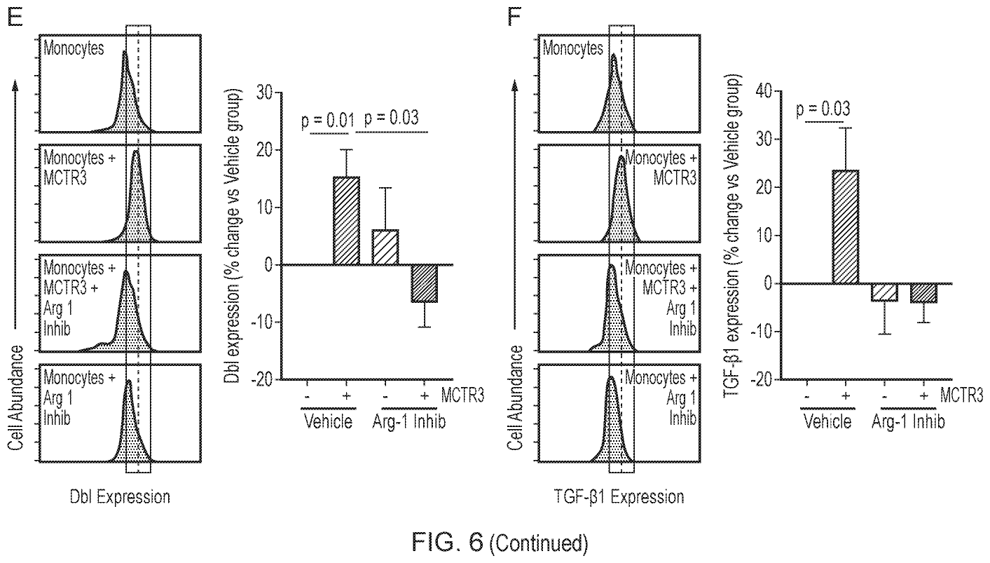

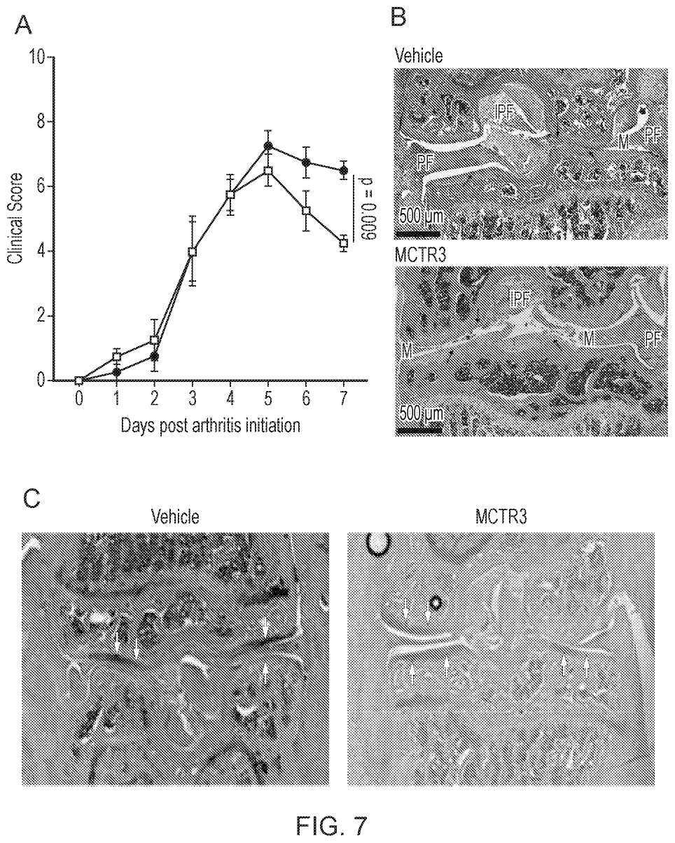

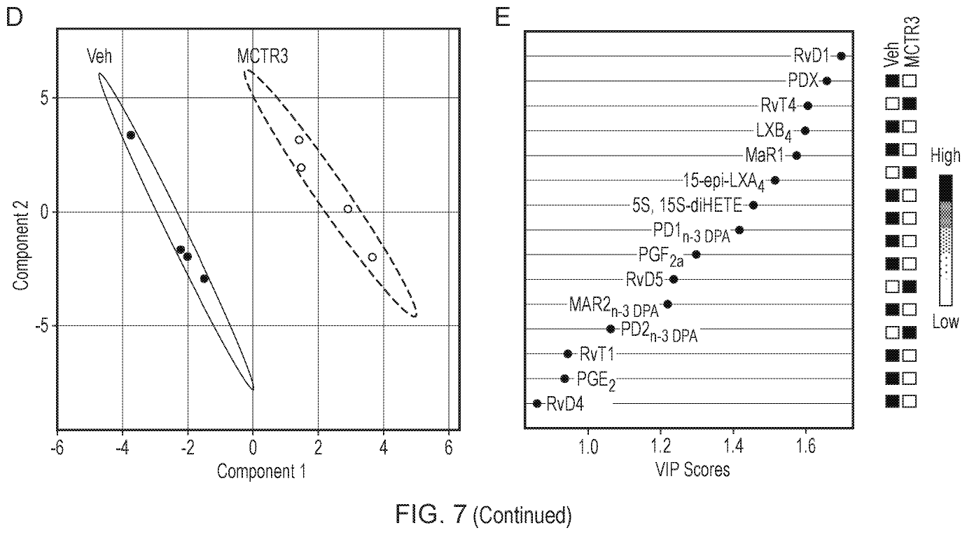

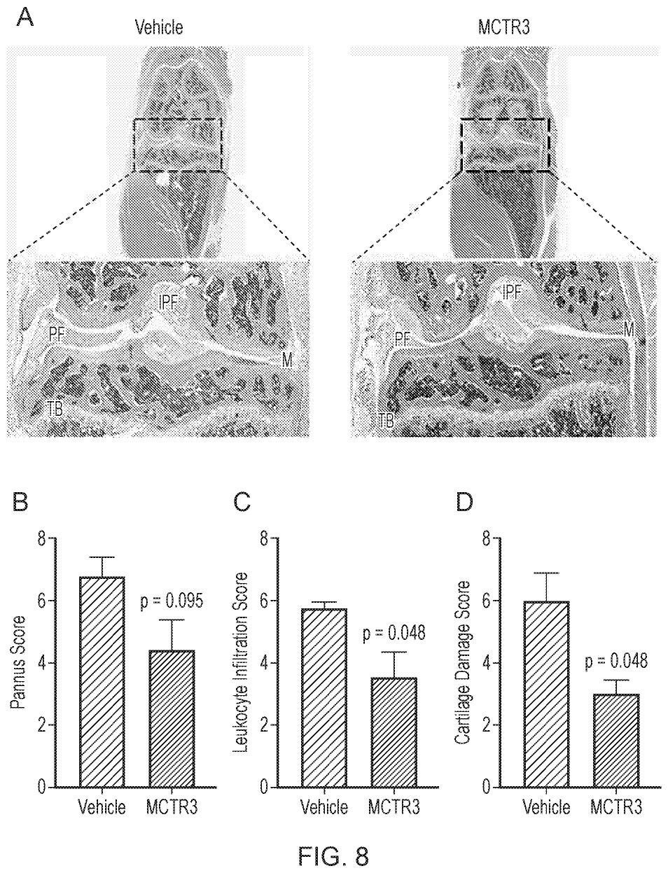

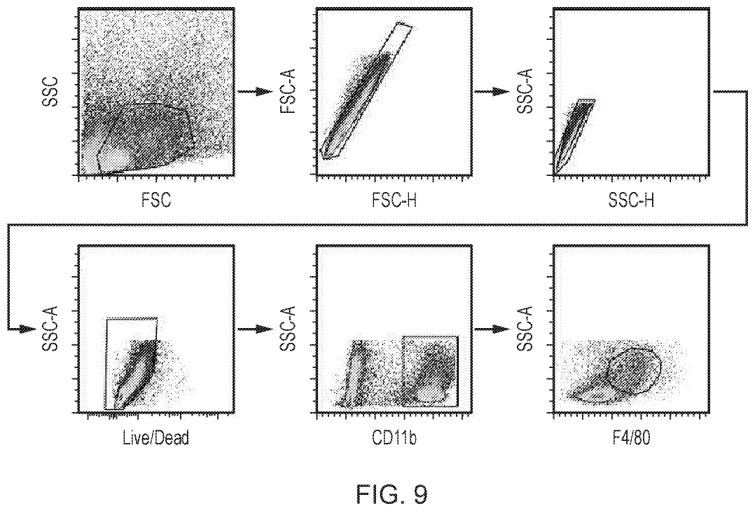

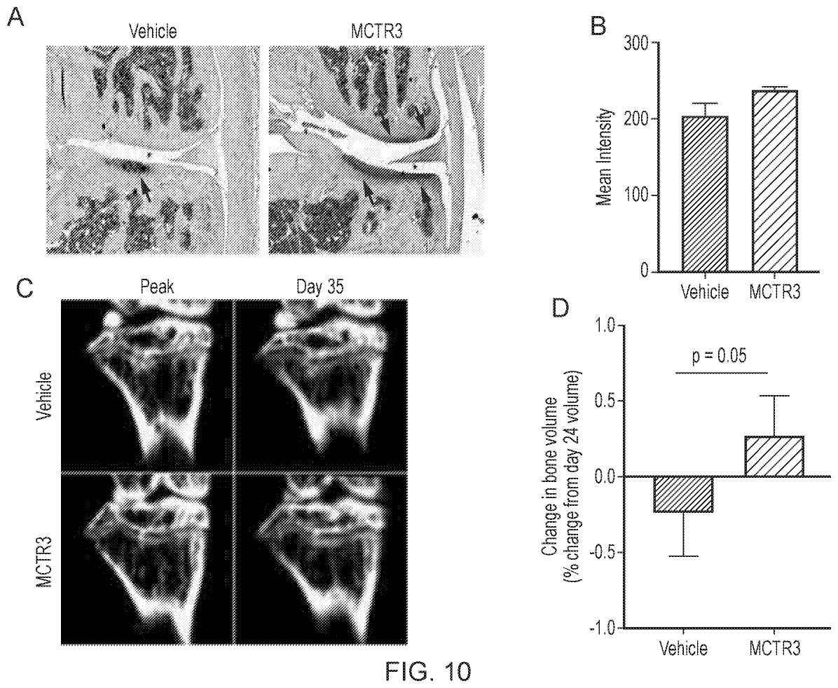

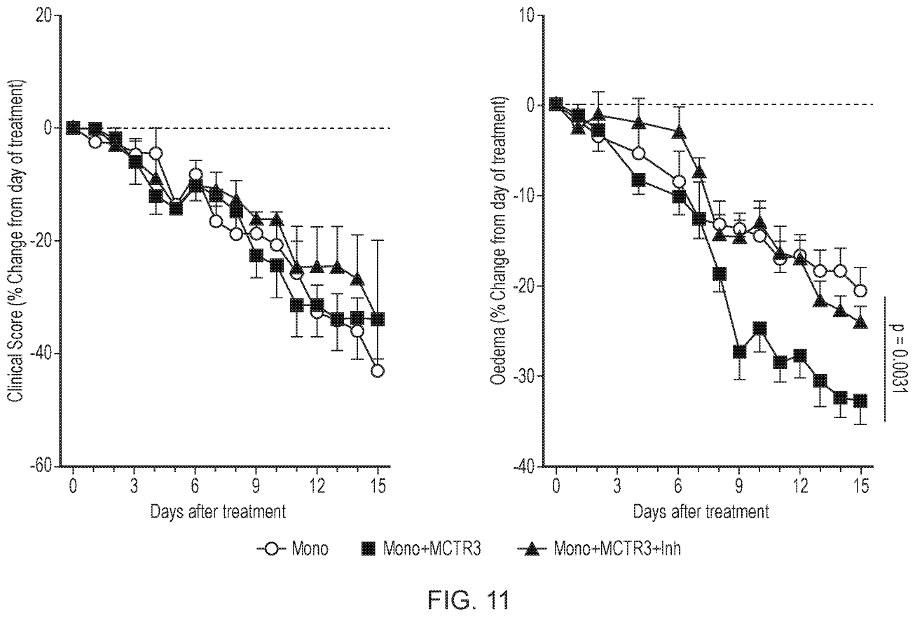

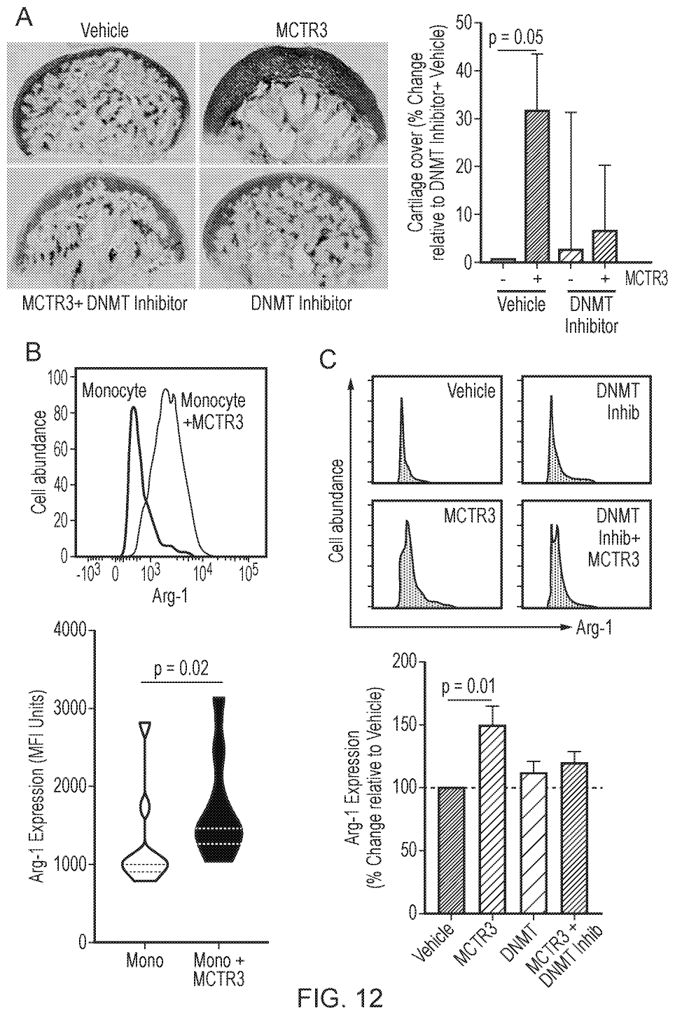

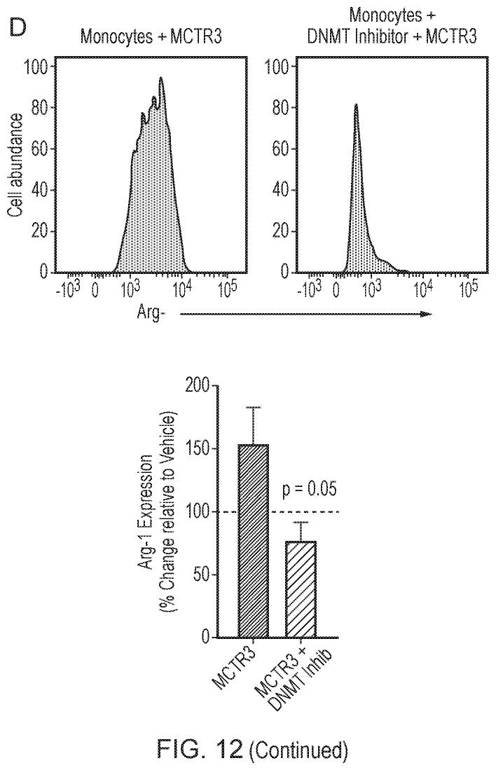

Recent studies suggest that changes in the epigenetic landscape of innate immune cells, including monocytes and macrophages, leads to their long term reprograming. Having observed that MCTR3 treatment led to a shift in MDM phenotype, a reduction in disease severity and increased joint repair, we next queried whether the joint protective actions of MCTR3 were linked with the reprogramming of MDM. To test this hypothesis, we incubated bone-marrow derived monocytes from donor mice with 1 nM of MCTR3 (MCTR3-reprogrammed monocytes) or vehicle and after 90 minutes cells were washed and administered to arthritic mice. Here we observed a reduction in disease severity in mice treated with MCTR3-reprogrammed monocytes as demonstrated by a significant reduction in oedema when compared with mice treated with monocytes incubated with vehicle alone (FIG. 3A, B).

To further evaluate the ability of MCTR3 to reprogram monocytes during inflammatory arthritis, we next evaluated whether the monocyte-derived activities of MCTR3 were retained in monocytes from arthritic mice. For this purpose, we isolated monocytes from the bone marrow of arthritic mice, treated them with MCTR3 or vehicle, administered them to recipient mice and evaluated joint inflammation. Here we found a reduction in both clinical scores and oedema volume in mice treated with MCTR3-reprogrammed monocytes when compared with mice given monocytes that were incubated with vehicle alone (FIG. 3C, D). The protective activities exerted by MCTR3-reprogrammed monocytes were observed at a histological level, where H&E staining revealed a significant reduction in leukocyte infiltration into the inflamed paws in mice treated with MCTR3-reprogrammed monocytes (FIG. 3E).

We also observed that MCTR3-reprogrammed monocytes regulated joint lipid mediator concentrations as observed by a shift in the cluster representing lipid mediator profiles obtained from joints of mice treated with these cells compared with mice treated with monocytes incubated with vehicle only (FIG. 3F). Notably, assessment of the top 15 mediators differentially regulated between the two groups demonstrated a marked upregulation of several joint protective SPM, including RvD1, in paws from mice receiving MCTR3-reprogrammed monocytes suggesting that these cells exert potent pro-resolving activities (FIG. 3G). Together, these findings indicate that MCTR3-reprogrammed monocytes exerted pro-resolving activities during inflammatory arthritis.

We next evaluated whether these cells also regulated tissue repair in arthritic mice. For this purpose, we assessed glycosaminoglycan content using Safranin-O staining in articular cartilage. This analysis demonstrated a significant increase in Safranin-O staining in mice treated with MCTR3-reprogrammed monocytes when compared with mice that were treated with monocytes alone (FIG. 4A). This increase in cartilage cover was linked with a significant increase in the expression of collagen 2 and collagen X in mice treated with MCTR3-reprogrammed monocytes (FIG. 4B,C).

To evaluate the mechanisms that lead to both the reduction in inflammatory arthritis and the upregulation of reparative mechanisms, we next evaluated the expression of molecules known to be involved in the propagation of inflammation and in the regulation of joint repair. We first evaluated the expression of Tumour necrosis factor (TNF)-α, matrix metalloproteinase (MMP) 7 and Fos-related antigen (Fra)-1 (Hannemann et al., 2019, J Clin Invest 129, 2669-2684). While Mmp7 expression in arthritic paws from both groups was essentially similar, the expression of both Tnf-α and Fra-1 was decreased in arthritic paws from mice treated with MCTR3-reprogrammed monocytes, reaching statistical significance for Tnf-α (FIG. 4D-F). Having observed a significant regulation of Tnf-α in mice receiving the reprogrammed monocytes, we next evaluated the expression of downstream targets of TNF-α which are known to regulate the Wnt signalling pathway, a key pathway in both bone and cartilage maintenance. For this purpose, we assessed the expression of Dickkopf (Dkk)-1, Lymphoid Enhancer Factor (LEF)-1 (Elayyan et al., 2017, FASEB J 31, 3116-3125), and Secreted frizzled-related protein (sFRP)-1 (Trenkmann et al., 2011, Ann Rheum Dis 70, 1482-1488). This analysis demonstrated that while Lef-1 and sFrp-1 expression was essentially similar between the two groups, Dkk-1 expression was significantly downregulated in mice treated with MCTR3-reprogrammed monocytes when compared with mice receiving monocytes alone (FIG. 4 G-I). Together these findings indicate that MCTR3-reprogrammed monocytes activate reparative mechanisms linked with both bone and cartilage repair.

### MCTR3 Reprograms the Arthritic Monocyte-Derived Macrophage Transcriptome

We next sought to determine the mechanism(s) by which MCTR3 elicited its protective actions. Having observed that short term incubation of monocytes with MCTR3 led to long term protective actions in arthritis we next queried whether this was at least in part linked the regulation of the epigenetic landscape of the cells. Given the central role that DNA methyltransferases play in this process, we next tested whether inhibition of these enzymes would reverse the protective activities of MCTR3-reprogrammed monocytes. Indeed, while disease severity was significantly reduced in mice administered MCTR3-reprogrammed monocytes derived from arthritic mice, incubation of these cells with a DNA methyltransferase inhibitor (RG108), abolished the protective actions of MCTR3 as observed by a decrease in the ability of these cells to regulate joint inflammation. Of note, this reversal of the protective actions of MCTR3 following incubation with a DNA methyltransferase inhibitor was observed with both KB×N initiated arthritis (FIG. 5A, B) as well as in glucose-6-phosphate isomerase peptide driven arthritis FIG. 11). Together these findings indicate that MCTR3 regulates circulating monocyte responses to limit joint inflammation and promote joint repair in a DNA methyltransferase-dependent manner.

To further explore the mechanism activated in MCTR3-reprogrammed monocytes that contribute to the observed protective actions we next incubated monocytes as detailed above and administered them to arthritic mice. After 10 days we collected the joints sorted the leukocytes and subjected these cells to single cell RNA sequencing. Assessment of transcript expression differences between cells isolated from mice receiving MCTR3-reprogrammed monocytes and those from mice receiving monocytes incubated with vehicle demonstrated that of the different cell subsets identified the biggest changes in transcript levels were observed in MDM with 63 differentially regulated genes (FIG. 5C, D and Table 6). Notably, out of these differentially expressed genes, Arginase-1 (Arg-1) was the gene that was upregulated to the greatest extent in cells isolated from mice receiving MCTR3-reprogrammed monocytes (FIG. 5D). Network analysis of genes that were found to be differentially regulated in MDM from MCTR3-reprogrammed monocytes demonstrated a differential regulation of genes linked with several processes involved in joint repair including osteoclast differentiation, arginine and proline metabolism, and extracellular matrix re-organization (FIG. 5E).

To further explore the mechanisms regulated by MCTR3 to reprogram mononuclear phagocytes we next evaluated the signaling pathways activated by MCTR3 using a phosphoproteomic approach. Gene ontology analysis of proteins found to be differentially phosphorylated in mononuclear phagocytes incubated with MCTR3 versus those incubated with vehicle alone demonstrated a marked regulation of proteins involved in post-transcriptional regulation and protein translation by MCTR3 (FIG. 5F and Table 7). This regulation was also observed when using the Kyoto Encyclopaedia of Genes and Genomes pathway database that highlighted an enrichment of spliceosome linked proteins as well as proteins involved in mRNA surveillance by MCTR3. (FIG. 5G and Table 7). In these studies, we also found that MCTR3 regulated the phosphorylation status of several proteins involved in both epigenetic and chromatin modification, including that of methylases Histone-lysine N-methyltransferase SETD2 as well as the deacetylases Histone deacetylase 1 and Histone deacetylase 2 (Table 7). These findings indicate that MCTR3 promotes the reprograming of mononuclear phagocytes by both regulating epigenetic programs and protein translation to facilitate the termination of inflammation and joint protection. They also support the hypothesis that MCTR3-reprogrammed monocytes recruited into arthritic joints exert their activities via the upregulation of tissue protective pathways.

In order to evaluate this hypothesis further, we employed an organ culture system whereby monocytes were obtained from the bone marrows of arthritic mice and incubated with or without MCTR3 then differentiated to macrophages for 5 days. We then incubated arthritic femur heads with these cells for 2 days and assessed their glycosaminoglycan. Assessment of Safranin-O staining demonstrated significantly higher glycosaminoglycan content in femur heads incubated with MDM obtained from MCTR3-reprogrammed monocytes (FIG. 12A). Of note, inhibition of methyltransferase activity reversed these protective actions of MCTR3-reprogrammed monocytes (FIG. 12A). Thereby, these findings lend support to the hypothesis that MCTR3 reprograms monocyte responses resulting in MDM that display joint protective activities.

TABLE 6
Transcript expression of different cell subsets from
joint leukocytes from mice treated with
monocytes or MCTR3-reprogrammed monocytes

| | | Log2(Fold | Adjusted |
|---|---|---|---|
| Cell type | Gene | Change) | p-value |
| Monocyte-derived | Arg1 | 3.00466 | 4.6E−09 |
| macrophages | | | |
| | Fabp5 | 1.69093 | 1.1E−08 |
| | Cstb | 1.10964 | 1.7E−08 |
| | Pi16 | −2.2344 | 3.9E−07 |
| | Hspa1b | −1.7295 | 6.1E−07 |
| | Osm | 1.58686 | 2E−05 |
| | Slc27a1 | 1.01616 | 5.1E−05 |
| | Nupr1 | −1.1402 | 0.00012 |
| | Ccl5 | −1.6147 | 0.0002 |
| | Gsn | −1.489 | 0.00057 |
| | Ebf1 | −2.3734 | 0.0006 |
| | Spp1 | 2.66477 | 0.00063 |
| | Ace | −1.4074 | 0.00165 |
| | Dpt | −2.2469 | 0.00289 |
| | Pam | −1.2786 | 0.00382 |
| | Selenom | −1.0473 | 0.00461 |
| | Ogn | −1.3442 | 0.00466 |
| | Atp2a1 | 1.48548 | 0.00506 |
| | Sod3 | −1.0363 | 0.00554 |
| | Rgs1 | 1.20654 | 0.00554 |
| | Plpp3 | −2.1638 | 0.0059 |
| | Fcgr4 | −1.2763 | 0.00629 |
| | Tnni2 | 1.32297 | 0.00629 |
| | Cavin1 | −1.2185 | 0.00678 |
| | Meg3 | −1.8147 | 0.00695 |
| | Tpm2 | 2.14035 | 0.00737 |
| | Tmem100 | −1.3947 | 0.00737 |
| | Tnxb | −2.2936 | 0.00737 |
| | Ifit1 | −1.0403 | 0.00737 |
| | Tpm1 | 1.40972 | 0.00737 |
| | Cd300e | −1.007 | 0.00784 |
| | Basp1 | 1.01228 | 0.00836 |
| | Hes1 | −1.7469 | 0.0093 |
| | Acta1 | 3.67159 | 0.00931 |
| | Retnla | −2.5315 | 0.00934 |
| | Ugdh | −1.0453 | 0.01571 |
| | Pacsin1 | 1.01914 | 0.01811 |
| | C1ra | −1.2313 | 0.01999 |
| | Nid1 | −1.496 | 0.02183 |
| | Cxcr6 | 1.15986 | 0.02183 |
| | Rnase4 | −1.1307 | 0.02205 |
| | Pcolce2 | −1.8447 | 0.02267 |
| | Fndc1 | −1.4321 | 0.02378 |
| | Cxcl2 | 1.20798 | 0.02426 |
| | Slc7a11 | 1.18246 | 0.02495 |
| | Sema3c | −1.0587 | 0.0282 |
| | Fabp3 | 1.00696 | 0.0282 |
| | Inmt | −1.4963 | 0.0282 |
| | Dpep1 | −1.3647 | 0.02872 |
| | Col6a2 | −1.3697 | 0.02879 |
| | Tnnc2 | 1.95338 | 0.02879 |
| | Treml4 | −1.0248 | 0.02995 |
| | Col6a1 | −1.7234 | 0.03058 |
| | Fstl1 | −2.3178 | 0.03136 |
| | Il1rn | 1.22401 | 0.03257 |
| | Tnnt3 | 2.08325 | 0.0335 |
| | Car1 | −1.4484 | 0.03461 |
| | Htra1 | −1.2523 | 0.03572 |
| | Mylpf | 1.45788 | 0.03821 |
| | Ccdc80 | −2.3691 | 0.03838 |
| | Hspa1a | −1.0763 | 0.03924 |
| | Fbn1 | −1.6012 | 0.04259 |
| | Ckm | 1.85215 | 0.04548 |
| B-cells | Igll1 | 1.98814 | 6.8E−08 |
| | Tuba1b | 1.15816 | 1.3E−07 |
| | Vpreb1 | 1.93786 | 6.4E−07 |
| | Dntt | 1.34604 | 2E−06 |
| | Stmn1 | 1.36397 | 0.00263 |
| | Pclaf | 1.29237 | 0.04659 |
| Myeloid cells | Igkc | 1.12351 | 2E−20 |
| T-cells | Hbb-bs | 5.28205 | 1.1E−42 |
| | Hba-a1 | 5.41246 | 5.9E−14 |
| | Hba-a2 | 4.50298 | 8.3E−12 |
| | Hbb-bt | 4.35532 | 8.8E−12 |
| | Acta1 | −3.7747 | 4.8E−11 |
| | Myl1 | −2.705 | 6.7E−09 |
| | Tnnc2 | −2.3889 | 2.9E−08 |
| | Prss34 | 3.01044 | 8.3E−08 |
| | Tpm2 | −1.8885 | 3E−07 |
| | Pvalb | −1.7228 | 6.2E−07 |
| | Tpm1 | −1.599 | 1.4E−06 |
| | Tnnt3 | −2.3095 | 2E−06 |
| | Tnni2 | −1.5296 | 1.7E−05 |
| | Ckm | −1.9523 | 3.7E−05 |
| | Mcpt8 | 2.42163 | 0.00044 |
| | Mylpf | −2.4847 | 0.00063 |
| | Dcn | −1.1157 | 0.00073 |
| | Gsn | −1.152 | 0.00145 |

### Arg-1 Mediates the Joint Protective Activities of MCTR3-Reprogrammed Monocytes

Having observed an upregulation of Arg-1 in joint macrophages from mice treated with MCTR3 (FIG. 1) and those treated with MCTR3-reprogrammed monocytes (FIG. 5) we next questioned whether Arg-1 was responsible for the observed protective activities exerted by these cells. For this purpose, we repeated the in vivo experiments detailed above, this time labelling the monocytes isolated from arthritic mice with a fluorescent membrane dye, PKH67, to differentiate them from endogenous monocytes. After 10 days we harvested the paws, liberated cells and assessed the expression of Arg-1 in PKH67⁺ MDM. Here we observed a significant increase in Arg-1 expression in PKH67⁺ MDM from mice that received MCTR3-reprogrammed monocytes when compared with those that received monocytes incubated with vehicle alone (FIG. 12B). This observation was also in MDM differentiated in vitro from MCTR3-reprogrammed monocytes and previously found to exert cartilage protective activities in our organ culture system (FIG. 6C). Notably, incubation of monocytes with a methyltransferase inhibitor reversed the ability of MCTR3 to upregulate this joint protective enzyme both in vitro and in vivo (FIG. 6C, D).

To further investigate the role of Arg-1 in mediating anti-arthritic and reparative activities of MCTR3-reprogrammed monocytes we used an siRNA approach to knockdown the expression of this enzyme in MDM and then evaluated the ability of these cells to promote cartilage repair using the organ culture system described above. Here we found that while MDM obtained from MCTR3-reprogrammed monocytes and transfected with a control siRNA markedly increased glycosaminoglycan content in arthritic femur heads, transfection of cells with siRNA to Arg-1, significantly abrogated the cartilage protective actions of these cells (FIG. 6A,B).

We next tested whether inhibition of Arg-1 activity in vivo would also reverse the protective actions of MCTR3-reprogrammed monocytes. For this purpose, we treated mice that received MCTR3-trained monocytes with the Arg-1 inhibitor N^{ω}-hydroxy-nor-L-arginine (nor-NOHA) and assessed joint disease activity. Here we found that as observed in previous experiments, administration of MCTR3-reprogrammed monocytes led to a reduction in both clinical scores and joint oedema, when compared with those mice that received monocytes incubated with vehicle alone. Of note treatment of mice with the Arg-1 inhibitor reversed these joint protective actions of MCTR3-reprogrammed monocytes as observed by an increase in clinical scores and oedema (FIG. 6C-D). Furthermore, inhibition of Arg-1 activity also reversed the upregulation of the Rac1 guanosine triphosphate (GTP)-exchange factor Dbl, an enzyme implicated in mediating the pro-resolving actions of Arg-1 in MDM and TGF-β1, a morphogen involved in bone and cartilage maintenance and repair (FIG. 6E,F). Together these findings support a role for Arg-1 in mediating the joint protective activities of MCTR3-reprogrammed monocytes.

### Discussion

Despite the notion that joint damage in patients with RA leads to significant morbidity current therapeutic approaches in RA are ineffective at activating joint repair programs. In the present studies we found that MCTR3 concentrations were negatively correlated with markers of both systemic and joint inflammation in patients with RA. Administration of MCTR3, to arthritic mice not only accelerated the resolution of joint inflammation but also activated joint reparative programs. Assessment of the cellular mechanisms involved in the observed protective activities demonstrated that MCTR3 reprogrammed monocytes and upregulated a number of tissue protective mechanisms including Arg-1 expression. Inhibition of DNA methyltransferase activity or Arg-1 led to a reversal of both the anti-inflammatory and joint protective actions of

### MCTR3 During Inflammatory Arthritis.

Unremitting inflammation is a key component in the destruction of joint tissues. In many patients this leads to severe deformation of the articular bones. Whilst such deformations in large articular bones, such as hips and knees can be rectified via arthroscopic surgery, smaller joints such as those in the fingers cannot be rectified using these approaches resulting is severe disability in many patients with RA. To date, the only therapeutics that impinge on this process of joint destruction are anti-TNF therapies which have been found to limit the activation of synovial cells and the progression of tissue destruction. Nonetheless, these therapeutics do not activate reparative process, and therefore any damage that occurs, especially in those patients with advanced bone and joint destruction are likely to be permanent. Furthermore, not all patients treated with anti-TNF therapies go into remission, which results in further tissue damage. In the present studies we found that MCTR3 administration, using a therapeutic paradigm, potently limited clinical signs of joint inflammation in two distinct models of inflammatory arthritis. This reduction in joint inflammation was linked with the activation of joint reparative mechanisms as demonstrated by a significant upregulation in both collagen 2 and collagen X expression, increased cartilage cover, increased bone volume and decreased callus size in joints from mice treated with MCTR3. The anti-inflammatory activities of MCTR3 are also in line with observations made with other SPM, e.g. AT-RvD1, RvD3, MaR1 and the RvD precursor 17-HDHA.

Trained immunity is now appreciated to play a significant role in both host protection from pathogenic infections as well as in the propagation of inflammation in chronic inflammatory conditions. Underpinning trained immunity is a change in the DNA methylation status of the cell that leads to a shift in the cellular responses to subsequent inflammatory stimulus. Studies investigating this process in RA demonstrated that circulating CD14⁺ monocytes from these patients expressed increased basal CD11b expression and produced higher concentrations of IL-1B and IL-6 when stimulated ex vivo. Notably, recent findings demonstrated that incubation of human monocytes with etanercept and adalimumab downregulated the trimethylation of H3K4, H3K27, H3K36 and H3K79 in the CCL2 promoter region by decreasing the expression of the related methyltransferases WDR5 and Smyd2.

Notably, the process of trained immunity has to-date been primarily linked with the reprogramming of cells towards an activated, potentially pro-inflammatory status. Our findings indicated that MCTR3-trained monocytes exert both anti-inflammatory and tissue reparative activities as observed by a decrease in joint disease activity, an upregulation in collagen 2 and collagen X expression. These protective activities of MCTR3 on reprogramming monocyte responses were reversed when these cells were incubated with a DNAse methyltransferase inhibitor, underscoring a central role for epigenetic reprogramming in mediating the protective activities of MCTR3 on monocytes.

Thus the present findings suggest that MCTR3 changes the epigenetic landscape of trained monocytes from arthritic mice to upregulate tissue protective and pro-resolving pathways in these cells. This hypothesis is supported by findings made in our transcriptomic and phosphor-proteomic analysis. Whereby we found that MCTR3 regulates the phosphorylation status of proteins involved in epigenetic and chromatin regulation in mononuclear phagocytes. Furthermore, sc-RNA seq analysis of synovial leukocytes from mice receiving MCTR3-reprogrammed monocytes demonstrated a marked shift in the transcriptome of MDM with an upregulation of several immunoregulatory and host protective genes, including Arg-1. In the present studies we found that MCTR3-reprogrammed monocytes yield MDM with elevated expression of Arg-1 both in vivo and in vitro. This observation was linked with a downregulation of Fra-1 expression in arthritic paws from these mice. Notably, inhibition of Arg-1 expression or activity reversed both the anti-inflammatory and the joint reparative activities of MCTR3-reprogrammed monocytes. Thus, these findings establish a central role of this enzyme in mediating the protective activities of MCTR3 during inflammatory arthritis.

Results from the present studies indicate that circulating monocytes may represent a novel target population for cell-based therapeutics. Our findings from both the in vivo and organ culture models suggest that MCTR3-reprogrammed monocytes display enhanced protective activities over non-reprogrammed monocytes in facilitating both the resolution of joint inflammation and promoting the repair and regeneration of arthritic tissues.

In summation, the present studies demonstrate that MCTR3 reprograms monocytes that to differentiate to MDM with anti-inflammatory and joint reparative properties. These anti-arthritic activities of MCTR3-reprogrammed monocytes were found to be mediated by the upregulation of Arg-1, whereby knockdown of this enzyme or inhibition of its activity reversed the abilities of these cells to reduce joint inflammation and repair arthritic joints. Together these observations uncover novel processes whereby MCTR3 governs monocyte responses in chronic inflammatory conditions.

### Materials and Methods

### RA Patient Samples

Plasma samples were taken from RA patients who were DMARDs and steroid-naive, had symptoms duration <12 months, and fulfilled the ACR/EULAR 2010 classification criteria for RA and recruited into the Pathobiology of Early Arthritis Cohort (PEAC http://www.peac-mrc.mds.qmul.ac.uk)). The PEAC cohort study was approved by the King's College Hospital Research Ethics Committee (REC 05/Q0703/198). Patients provided informed consent. Peripheral blood samples were obtained from patients recruited at Barts Health NHS Trust undergoing ultrasound (US)-guided synovial biopsy.

### Targeted Lipid Mediator Profiling

All samples were extracted using solid-phase extraction columns as in (Gomez et al., 2020, Nat Commun 11, 5420). In brief, samples were placed in ice-cold methanol containing deuterated internal standards (d₈-5S-HETE, d₄-LTB₄, d₅-LXA₄, d₄-PGE₂, d₅-RvD2, d₅-MaR1, d₅-MaR2, d₅-RvD3, d₄-RvE1, d₅-17R-RvD1, d₅-LTC₄, d₅-LTD₄ and d₅-LTE₄) representing each chromatographic region of identified LM. Following protein precipitation (−20° C. for a minimum of 45 min), samples were centrifuged and supernatants extracted using an ExtraHera System (Biotage) using solid-phase extraction with Isolute C18 500 mg columns (Biotage). Methyl formate and methanol fractions were collected, brought to dryness and resuspended in phase (methanol/water, 1:1, vol/vol) for injection on a Shimadzu LC-20AD HPLC and a Shimadzu SIL-20AC autoinjector, paired with a QTrap 5500 or QTrap 6500⁺ (Sciex). In the analysis of mediators eluted in the methyl formate fraction, an Agilent Poroshell 120 EC-C18 column (100 mm×4.6 mm×2.7 μm) was kept at 50° C. and mediators eluted using a mobile phase consisting of methanol/water/acetic acid of 20:80:0.01 (vol/vol/vol) that was ramped to 50:50:0.01 (vol/vol/vol) over 0.5 min and then to 80:20:0.01 (vol/vol/vol) from 2 min to 11 min, maintained till 14.5 min and then rapidly ramped to 98:2:0.01 (vol/vol/vol) for the next 0.1 min. This was subsequently maintained at 98:2:0.01 (vol/vol/vol) for 5.4 min, and the flow rate was maintained at 0.5 ml/min. QTrap 5500 was operated in negative ionization mode using a multiple reaction monitoring method. In the analysis of mediators eluted in the methanol fraction, an Agilent Poroshell 120 EC-C18 column (100 mm×4.6 mm×2.7 μm) was kept at 50° C. and mediators eluted using a mobile phase consisting of methanol/water/acetic acid 55:45:0.5 (vol/vol/vol) over 5 min, that was ramped to 80:20:0.5 (vol/vol/vol) for 2 min, maintained at 80:20:0.5 (vol/vol/vol) for the successive 3 min and ramped to 98:2:0.5 (vol/vol/vol) over 3 min. This condition was kept for 3 min. QTrap 6500⁺ was operated in positive ionization mode using a multiple reaction monitoring method. Each lipid mediator was identified using established criteria, including: (1) presence of a peak with a minimum area of 2000 counts, (2) matching retention time to synthetic or authentic standards, (3) ≥5 data points, and (4) matching of at least 6 diagnostic ions to that of reference standard, with a minimum of one backbone fragment being identified. Calibration curves were obtained for each mediator using synthetic compound mixtures at 0.78, 1.56, 3.12, 6.25, 12.5, 25, 50, 100, and 200 pg that gave linear calibration curves with an r² values of 0.98-0.99

### Animal Studies

10-week-old C57BL/6 mice (Charles River, UK), DBA/1 mice (Charles River, UK) and C57BL/6-Ly5.1 mice (Charles River, Italy) were used in the reported studies. UK Home Office regulations (Guidance on the Operation of Animals, Scientific Procedures Act, 1986) and Laboratory Animal Science Association (LASA) Guidelines (Guiding Principles on Good Practice for Animal Welfare and Ethical Review Bodies, 3rd Edition, 2015) were strictly adhered. Mice were kept in specific pathogen free housing, food and water were provided ad libitum and kept with a 12h light-dark cycle, with lights on between 7:00 h and 19:00h.

### Inflammatory Arthritis

G6PI peptide induced arthritis: Antigen DBA/1 mice were immunised with a G6PI emulsion (120 μL/mouse), prepared by sonication of 10 μg G6PI peptide (Sequence: IWYINCFGCETHAML; Cambridge Peptides Ltd.) in 50 μL complete Freund's adjuvant (CFA) and 50 μL DPBS^{-/-} per mouse (Schubert et al., 2004, J Immunol 172, 4503-4509), via intradermal injection at the base of the tail to initiate inflammatory arthritis. Arthritic DBA/1 mice were treated with 1 μg/mouse MCTR3 or vehicle (DPBS^{-/-}0.1% EtOH) on day 24, 26 and 28 intravenously (i.v.). In separate experiments, 5×10⁵ bone marrow (BM) derived monocytes from naive mice previously incubated at 37° C. with either vehicle (DPBS^{-/-}+0.1% DMSO) or 10 μM RG108 (Sigma), a DNA methyltransferase (DNMT) inhibitor, for 15 min and then with a vehicle (DPBS^{-/-}+0.1% EtOH) or 1 nM MCTR3 for 90 minutes were administered i.v. (120 μL/mouse) to arthritic DBA mice on day 24. Paws were collected for microCT analysis and flow cytometry on day 36.

K/B×N serum induced arthritis: Arthritogenic K/B×N serum (100 μL/mouse) was administered via intraperitoneal (i.p.) injection to C57BL/6 mice on day 0 and 2 to induce self-resolving inflammatory arthritis (Norling et al 2016, JCI Insight 1, e85922). Disease severity was evaluated using a 26-point arthritic scoring system and ankle pad oedema was measured daily using callipers (Flak et al., 2019, JCI Insight 4). For femur head collection and BM cell isolations for in vitro cell cultures, mice were culled on Day 5. Otherwise, mice were administered a third K/B×N serum injection on either day 8 or 9 to prolong inflammatory arthritis. Mice were then treated i.v. with vehicle (DPBS^{-/-}+0.1% EtOH) or 1 μg/mouse MCTR3 on day 10, 12 and 14 and on day 25, paws were collected for histology, single cell RNA sequencing and flow cytometry and blood was collected for ELISAs.

In separate experiments, arthritis was initiated and prolonged as detailed above. On day 12, mice were treated via i.v injection with 2×10⁶ BM derived monocytes, obtained from arthritic C57BL/6 mice that were isolated and trained as detailed below. Paws were collected for flow cytometry, single cell RNA sequencing and histology on day 22.

In other experiments, arthritis was initiated and prolonged as above, on day 12 they were treated with vehicle or 200 μg N^{ω}-Hydroxy-nor-L-arginine (nor-NOHA), an arginase 1 inhibitor, administered via i.p. injections daily. On Day 22 paws were collected for flow cytometry and histology.

### Bone Marrow Isolations

Bone marrow cells were collected from naive DBA/1 mice, arthritic C57BL/6 mice on day 5 after the initial K/B×N injection or arthritic C57BL/6 mice on day 12 after the initiation of arthritis (see above). Briefly, femurs, tibiae and humeri were placed in 70% EtOH and rinsed in PBS-1. The epiphysis were removed, and a 25G needle was used to flush the bone marrow with 2 mL PBS/per bone. Cells were dispersed gently with a 19G needle, filtered through a 70 μM strainer, centrifuged at 400×g for 5 minutes at 4° C. and suspended in DPBS^{+/+}.

For monocyte adoptive transfer experiments, bone marrow-derived monocytes were the isolated using EasySep™ Mouse Monocyte Isolation Kit (STEMCELL) according to manufacturer's instructions. Isolated monocytes from arthritic C57BL/6 mice were labelled with PKH67 Red Fluorescent Cell Linker kit (Sigma), following manufacturer's instructions. Monocytes were then incubated with either vehicle (DPBS^{+/+}+0.01% EtOH) or 1 nM MCTR3 for 90 min at 37° C. In separate experiments monocytes were first incubated with vehicle (DPBS^{+/+}+0.1% DMSO) or 10 μM RG108 (Sigma) for 15 min, prior to incubation with MCTR3 (1 nM) or vehicle (DPBS^{+/+}+0.01% EtOH; 37° C.).

In other experiments bone marrow cells were isolated from long bones collected from arthritic mice 5 days after the initiation of arthritis and seeded into 10 cm dishes. These were then incubated at 37° C. for 45 minutes in PBS+/+, the supernatant was removed and cells were washed with PBS^{-/-} to remove non-adherent cells. Adherent cells were incubated with either 10 μM RG108 or a vehicle (DPBS^{+/+}+0.1% DMSO) for 45 minutes in 5 mL DMEM containing 1% penicillin and streptomycin (P/S), following which, 1 nM MCTR3 or vehicle (DPBS^{+/+}+0.1% EtOH) was added to the media. After 2 hours, an additional 5 mL DMEM containing 1% P/S and 0.2% FBS (for a final concentration of 0.1% FBS) was added and the cells were incubated at 37° C. at 5% CO₂ for a further 22 hours. Media was replaced with DMEM containing 1% P/S, 10% FBS and 20 ng/ml murine GM-CSF, and incubated for a further 4 days to allow for macrophage differentiation. Subsequently, macrophages were detached using 5 mM EDTA in PBS^{-/-} and seeded, at 1.5×10⁵ cells/well, into 24-well Transwell plates in DMEM containing 1% P/S and 10% FBS for co-incubations with femoral heads. In separate experiments, BM derived monocytes were treated as described above, and following the replacement of media with DMEM containing 1% P/S, 10% FBS and 20 ng/ml murine GM-CSF, monocytes were allowed to differentiate for a further 6 days. Media was refreshed after 3 days.

To evaluate the role of Arg-1 in mediating the joint protective actions of monocyte derived macrophages, bone marrow monocytes were incubated with vehicle (PBS+0.1% EtOH) or 1 nM MCTR3 in 5 mL DMEM containing 1% P/S for 2 hours at 37° C. at 5% CO₂, after which an additional 5 mL DMEM containing 1% P/S and 0.2% FBS (for a final concentration of 0.1% FBS) was added and the cells were incubated for a further 22 hours. Media was then replaced with DMEM containing 1% P/S, 10% FBS and 20 ng/ml murine GM-CSF and incubated for a further 2 days. Adherent cells were detached with 5 mM EDTA in PBS^{-/-} and seeded into 24-well Transwell plates, at 2×10⁵ cells/well. Cells were then incubated in serum-free Accell siRNA delivery medium containing either 1 μM Accell anti-mouse Arg1 siRNA SMARTpool or mouse control siRNA (Dharmacon) at 37° C. at 5% CO₂ for 48 hours. Cells were washed with PBS^{-/-} and DMEM containing 1% P/S and 10% FBS was added to the cells for co-incubations with femur heads.

### Femur Head Isolation and Culture

Femur heads were collected as described in Headland et al. (2015, Sci Transl Med. 2015; 7(315):315ra190). Femur heads were removed from arthritic C57BL/6 mice, washed in 70% EtOH, then in DPBS^{-/-}, and incubated in 200 μL pre-warmed serum-free DMEM containing high glucose and 1% insulin-transferrin-selenium for 48 hours at 37° C. and 5% CO₂. The medium was replaced with DMEM containing 10% FBS and 10 ng/ml IL-1B and femur heads were incubated for a further 72 hours. These were then co-incubated with MDM that were prepared as detailed above for 48 hours at 37° C. and 5% CO₂. Tissues were then collected and fixed in 10% neutral buffered formalin (NBF) for histology.

### Elisas

Mouse Procollagen I N-Terminal Propeptide (PINP; Abbexa; 4× dilution) and Cross Linked C-Telopeptide of Type-I Collagen (CTXI; Abbexa; 4× dilution) were evaluated in plasma collected from arthritic C57BL/6 mice treated with vehicle or MCTR3, as per manufacturer's instruction.

### Histology

The femur heads and joints were fixed in 10% NBF, respectively, for 72 hours and decalcified in 10% EDTA (w/v) in PBS^{+/+} for 2 weeks with shaking. The decalcified tissue was then processed and embedded in paraffin and 4-micron sections were cut.

### Safranin O

To assess cartilage deposition, tissues were incubated for 5 minutes with 0.1% Safranin O in 0.2 M acetic acid and 0.2 M sodium acetate, pH 4, washed in dH₂O for 2 minutes and air-dried. For counter-staining, 0.05% Light-green (GeneTex) in dH₂O was added to the sections for 3 minutes and washed with dH₂O twice for 2 minutes each. Sections were incubated twice in 100% EtOH for 5 minutes each, briefly dipped in Histoclear, left to air dry and mounted with Entellan. Safranin O staining was imaged using either the EVOS microscope or Nanozoomer Slide scanner and NDP.view 2 software (Hamamatsu Photonics) and assessed with ImageJ 1.53.

### Collagen

Slides were heated at 50° ° C. for 30 minutes, incubated in Histoclear twice, then twice in 100% EtOH for 5 minutes each. Sections were washed in dH₂O for 1 minute, air dried at room temperature, fixed in 4% PFA for 5 minutes and washed in PBS with on an orbital shaker for two 5 minutes intervals. For digestion of pepsin, slides were incubated in 0.02% HCl for 7 minutes at 37° C., then for 20 minutes at 37° C. in 3 mg/mL pepsin solution in 0.02% HCl equilibrated to 37° C. This was washed twice in PBS for 5 minutes on an orbital shaker, quenched by incubating in 50 nM ammonium chloride twice, each for 5 minutes on an orbital shaker. Sections were washed as above and blocked in 20% FBS in PBS for 1 hour at room temperature. Sections were incubated with primary mouse polyclonal anti-collagen type II (Merck Millipore; 1:500 in 20% FBS in PBS) for 1 hour at room temperature in the dark, washed 3 times in PBS for 10 minutes and incubated with secondary AF488 Goat anti-mouse IgG (1:400 in 20% FBS in PBS) for 1 hour at room temperature. This was washed in PBS 3 times in the dark for 10 minutes each on an orbital shaker and incubated in the dark with efluoro570 anti-collagen X (1:200 in 20% FBS in PBS) at 4° C. overnight. PBS was used to wash the sections 3 times for 10 minutes each on an orbital shaker in the dark and slides were mounted with Mowiol with DAPI overnight.

### Micro-CT Scans

The Siemens INVEON® PET/CT scanner (Siemens Preclinical Solutions, Knoxville, TN) with the Inveon Acquisition Workplace software was used perform micro-CT scans of the arthritic DBA mice knees at peak of disease on Day 24 and following resolution of inflammatory arthritis, on day 35. All procedures were done in accordance with UK Home Office Regulations. Before scanning, the center offset and light/dark calibration was performed and a new workflow was created on the scanner. Mice were anesthetised with 3-5% inhalation anaesthesia, which was reduced to and maintained at 1.5% during scanning, at a rate of 1.5 L/min. Mice were laid in prone position on a heating pad at 37° C. to maintain body temperature during scanning. Scanning was performed at a voltage of 70 kV, using an X-ray current of 500 μA and at an exposure time of 2000 ms/projection for 360 projections. Hounsfield correction was used for image reconstruction.

To evaluate bone callus arthritic joints were collected 25 days after initiation of arthritis using K/B×N serum. Samples were wrapped in plastic film prior to scanning to prevent drying and scanned using a Skyscan 1172F (Bruker, Kontich, Belgium). The X-ray source was operated at 50 kV and 200 μA, using an Aluminium 0.5 mm filter and an exposure time 960 ms using a voxel size of 5 μm. Projection images were reconstructed into tomograms using NRecon 1.7.3.1 (Bruker, Kontich, Belgium) and repositioned using Dataviewer 1.5.4 (Bruker, Kontich, Belgium) with bone analysis performed in CTAn 1.18.4 (Bruker, Kontich, Belgium). Volume rendered 3D visualisations were created using CTVox 3.3 (Bruker Kontich, Belgium).

Leukocyte Isolation from Arthritic Paws

Hind paw tissue digestion to isolate leukocytes from arthritic joints was performed as described in Norling et al., (2016, JCI Insight 1, e85922). Briefly, following the removal of skin and muscle, the hind paw was incubated in 15 mL digestion buffer (RPMI containing 0.5 μg/mL collagenase D and 40 μg/mL DNAse) at 37° C. for 30 minutes with vigorous agitation. Liberated cells within the digestion buffer were passed through a 70 μM strainer into 10 mL 10% FBS in RMPI on ice. The digestion incubation was repeated and the cell suspension volume was made up to 50 mL with 10% FBS in RMPI. Cells were centrifuged at 400×g for 10 minutes at 4° C. and suspended in PBS for flow cytometry.

### Gene Expression

Tissue was homogenised using a BeadBeater and an RNeasy Mini Kit (Qiagen) was used to extract RNA, as per manufacturers instruction. cDNA synthesis was achieved using Superscript II Reverse Transcriptase (Invitrogen), as per manufacturers instruction. QuantiTect Primer Assays (Qiagen) for mouse Tnf-α, Mmp7, Fra-1, Dkk1, Lef1 and sFrp-1 were used with SYBR green I fluorescent dye for real-time PCR (qRT-PCR) evaluation with the StepOne™ Real-Time PCR System (ThermoFisher). Target gene expression was expressed as a value relative to Actb expression.

### Flow Cytometry

Isolated cells from arthritic paws were incubated with the following fluorescently conjugated antibodies: PE mouse anti-mouse CD64 (Biolegend), PE-Cy5 rat anti-mouse CD11b (Biolegend) and APC/Cy-7 rat anti-mouse F4/80 (Biolegend) at a dilution of 1:100 in PBS^{-/-} with 0.02 BSA for 30 minutes at 4° C. Cells were incubated with BD Fixation/Permeabilisation buffer solution and then with BD Permeabilisation Buffer, each for 20 minutes at room temperature. Intracellular staining was performed by incubating cells with BV 421 anti-mouse TGF-31 (1:50 dilution, Biolegend), PE sheep anti-mouse Arginase 1 (1:50 dilution, R&D) and polyclonal rabbit anti-DBL (1:100 dilution, Cell Signaling Technologies) for 30 minutes at 4° C. Rabbit anti-DBL was conjugated PerCp/Cy5.5 using Abcam's PerCP/Cy5.5 Conjugation Kit, as per manufacturer's instructions. Cells were incubated with TruStain X to quench non-specific binding. Multiparameter analysis was performed with LSR Fortessa cell analyser (BD Biosciences) and analysed using FlowJo (Tree Star Inc., V10).

### Single-Cell 3′ RNA Sequencing

Following paw tissue digestion, as described above, live cells were obtained using EasySep™ Dead Cell Removal (Annexin V) kit (STEM CELL) according to manufacturer's instruction. Cells were incubated with AF700 CD45 (Biolegend) at a dilution of 1:100 in PBS^{-/-} with 0.02% BSA for 30 minutes at 4° C. Non-specific staining was blocked with TruStain X. Cells were suspended in PBS^{-/-} with 0.02% BSA and the BD FACS Aria II was used sort for CD45 positive cells, which were collected in PBS containing 0.1% BSA for single cell sequencing.

### Sample Quality Control

8 single cell suspensions were provided and were assessed for cell number using the Luna FL automated cell counter (Logos biosystems, South Korea). Cells appeared intact and well distributed with an average count of 148 cells/μL.

### Single-Cell Library Generation and RNA-Sequencing

An equivalent volume of 4000 cells was loaded to the 10× Chromium™ Single Cell A Chip (PN-1000009) using the Chromium™ 3′ Library & Gel Bead Kit v2 (PN-120267) as described in the manufacturers user guide (10× Genomics, California, USA). GEMs were recovered from the chip and appeared opaque and uniform in colour. 14 cycles of cDNA amplification were performed on the purified GEM-RT product, and cDNA was examined for quality using the Agilent 2200 Tapestation with the High-sensitivity D5000 screentape and reagents (Agilent Technologies, Waldbronn, Germany), and the Qubit® 2.0 Fluorometer and Qubit dsDNA HS Assay Kit (Life Technologies, California, USA). 35 of cDNA was used to prepare the 10×3'RNA libraries and 12 cycles were used for sample index PCR. Final cleaned libraries were quantified using the Qubit® 2.0 Fluorometer and Qubit dsDNA HS Assay Kit and average fragment size checked using the Agilent D1000 screentape and reagents.

The final pooled library was run on a NextSeq500 High-output v2.5 150-cycle kit with a 26[8]98 cycle configuration to generate 400 million read pairs in total. ScRNA-seq data generated during this study are available at the Gene Expression Omnibus (GSE174118)

### Preliminary Data Analysis

Raw sequence data was processed using the 10× Genomics cellranger pipeline (v2.2.0). Briefly, fastq files were generated for the sample, followed by barcode processing and alignment to the mm 10 genome reference using cellranger count.

### Differential Gene Expression Analysis

The Cell Ranger pipeline was used to analyze the data generated by the single cell RNA-seq (10× Genomics; https://support.10× genomics.com/single-cell-gene-expression/software/). Shortly, the pipeline demultiplexes raw base call files generated by Illumina sequencers in FASTQ files and then aligns, filters and counts (barcode and UMI) the reads. The alignment was done using STAR (https://github.com/alexdobin/STAR) and the Mus musculus genome (GRCm38) as the reference genome.

Before differential gene expression analysis different samples for each group were aggregated (to have a normalized set of cells per group) using “cell ranger aggr” function. t-SNE analysis were performed for clustering different cell types. Distinct leukocyte subsets were identified using K-means clustering and choosing the cluster with statistically different genes using the Loupe Cell Browser software (10× Genomics).

Differential gene expression analysis was performed using the likelihood ratio test from Edge R (Bioconductor R package; https://bioconductor.org/packages/release/bioc/html/edgeR.html). Good gene expression was considered if at least two cells contain more than 2 transcripts from the gene. Statistical significance was considered with an adjusted (Benjamini-Hochberg procedure correction) p-value <0.05.

### Pathway Enrichment Analysis

Pathway analysis was performed uploading the differentially expressed genes in NetworkAnalyst 3.0 (networkanalyst.ca/NetworkAnalyst/home.xhtml) and searching for the enriched pathways from KEGG (p value <0.05, Fisher exact test followed by multiple comparison correction using Benjamini-Hochberg procedure) database.

### Phosphoproteomic Analysis

For evaluation of signalling pathways activated by MCTR3, monocytes were isolated from peripheral blood of healthy volunteers, incubated with GM-CSF (20 ng/ml, in RPMI containing 10% human serum) for 7 days and then incubated with MCTR3 (1 nM, in DPBS^{+/+}).

Phosphoproteomics experiments were performed using mass spectrometry. In brief, cells were lysed in 8M urea buffer and supplemented with phosphatase inhibitors (10 mM Na₃VO₄, 100 mM B-glycerol phosphate and 25 mM Na₂H₂P₂O₇ (Sigma)). Proteins were digested into peptides using trypsin. Phosphopeptides were enriched from total peptides by TiO₂ chromatography. Dried phosphopeptides were dissolved in 0.1% TFA and analysed by nanoflow ultimate 3000 RSL nano instrument was coupled on-line to a Q Exactive plus mass spectrometer (Thermo Fisher Scientific). Gradient elution was from 3% to 35% buffer B in 120 min at a flow rate 300 nL/min with buffer A being used to balance the mobile phase (buffer A was 0.1% formic acid in water and B was 0.1% formic acid in acetonitrile). The spray voltage was 1.95 kV and the capillary temperature was set to 255° C. The Q-Exactive plus was operated in data dependent mode with one survey MS scan followed by 15 MS/MS scans. The full scans were acquired in the mass analyser at 375-1500m/z with the resolution of 70 000, and the MS/MS scans were obtained with a resolution of 17 500.

MS raw files were converted into Mascot Generic Format using Mascot Distiller (version 2.5.1) and searched against the SwissProt database (release December 2015) restricted to human entries using the Mascot search daemon (version 2.5.0). Allowed mass windows were 10 ppm and 25 mmu for parent and fragment mass to charge values, respectively. Variable modifications included in searches were oxidation of methionine, pyro-glu (N-term) and phosphorylation of serine, threonine and tyrosine.

### Statistics

GraphPad Prism 8 (GraphPad Software, La Jolla, CA, USA) was used to assess differences between the groups using Spearman test, one-sample Wilcoxon signed rank test for normalized data between 2 groups, Mann-Whitney U test between 2 groups, a One-way ANOVA between 3 groups or Two-way ANOVA for time course analysis.

All publications mentioned in the above specification are herein incorporated by reference. Various modifications and variations of the disclosed products, uses and methods of the invention will be apparent to the skilled person without departing from the scope and spirit of the invention. Although the invention has been disclosed in connection with specific preferred embodiments, it should be understood that the invention as claimed should not be unduly limited to such specific embodiments. Indeed, various modifications of the disclosed modes for carrying out the invention, which are obvious to the skilled person are intended to be within the scope of the following claims.

TABLE 7
KEGG Pathway, Reactome Gene Sets and GO Biological Processes from mononuclear phagocytes incubated with Vehicle or MCTR3.

| | | | | | | | Log(q- | | | |
|---|---|---|---|---|---|---|---|---|---|---|
| Analysis | GroupID | Category | Term | Description | LogP | −Log10P | value) | InTerm_InList | Genes | Symbols |
| Kegg | 1_Summary | KEGG | hsa03040 | Spliceosome | −7.66232 | 7.662315 | −5.056 | 14/134 | 4670, 4686, 6428, 6434, 8683, 9416, | HNRNPM, NCBP1, SRSF3, TRA2B, SRSF9, |
| | | Pathway | | | | | | | 10569, 10772, 11325, 22938, 22985, | DDX23, SLU7, SRSF10, DDX42, SNW1, |
| | | | | | | | | | 23451, 27316, 199746 | ACIN1, SF3B1, RBMX, U2AF1L4 |
| | 2_Summary | KEGG | M00002 | Glycolysis, core | −4.6043 | 4.604305 | −2.299 | 4/12 | 2597, 5223, 5315, 7167 | GAPDH, PGAM1, PKM, TPI1 |
| | | Pathway | | module | | | | | | |
| | | | | involving three- | | | | | | |
| | | | | carbon | | | | | | |
| | | | | compounds | | | | | | |
| | 3_Summary | KEGG | ko04330 | Notch signaling | −4.0393 | 4.039297 | −1.831 | 6/48 | 3065, 3066, 5664, 6868, 22938, 151636, | HDAC1, HDAC2, PSEN2, ADAM17, SNW1, |
| | | Pathway | | pathway | | | | | 1108, 1385, 5315, 5925, 6672, 8359, | DTX3L, CHD4, CREB1, PKM, RB1, |
| | | | | | | | | | 27044, 695, 960, 3383, 7431, 9641, | SP100, H4C1, SND1, BTK, CD44, |
| | | | | | | | | | 57506 | ICAM1, VIM, IKBKE, MAVS |
| | 4_Summary | KEGG | ko03015 | mRNA | −3.28372 | 3.283722 | −1.312 | 7/91 | 4686, 5411, 8106, 8189, 10250, | NCBP1, PNN, PABPN1, SYMPK, SRRM1, |
| | | Pathway | | surveillance | | | | | 22985, 51585, 1207, 5903, 9669, 54913 | ACIN1, PCF11, CLNS1A, RANBP2, |
| | | | | pathway | | | | | | EIF5B, RPP25 |
| | 5_Summary | KEGG | ko04144 | Endocytosis | −3.11665 | 3.116648 | −1.251 | 12/260 | 5371, 9135, 9815, 10095, 10564, 23362, | PML, RABEP1, GIT2, ARPC1B, ARFGEF2, |
| | | Pathway | | | | | | | 23527, 27183, 50807, 80223, 116988, | PSD3, ACAP2, VPS4A, ASAP1, |
| | | | | | | | | | 253725 | RAB11FIP1, AGAP3, WASHC2C |
| | 6_Summary | KEGG | M00050 | Guanine | −3.0343 | 3.034302 | −1.241 | 3/13 | 3614, 3615, 5315 | IMPDH1, IMPDH2, PKM |
| | | Pathway | | ribonucleotide | | | | | | |
| | | | | biosynthesis | | | | | | |
| | | | | IMP => | | | | | | |
| | | | | GDP, GTP | | | | | | |
| | 7_Summary | KEGG | ko05202 | Transcriptional | −2.70368 | 2.703684 | −1.049 | 9/180 | 1025, 2119, 2313, 3065, 3066, 5371, | CDK9, ETV5, FLI1, HDAC1, HDAC2, |
| | | Pathway | | misregulation | | | | | 7709, 9611, 55589 | PML, ZBTB17, NCOR1, BMP2K |
| | | | | in cancer | | | | | | |
| | 8_Summary | KEGG | ko04722 | Neurotrophin | −2.6025 | 2.602505 | −0.996 | 7/119 | 673, 2889, 4215, 5580, 5664, 6197, | BRAF, RAPGEF1, MAP3K3, PRKCD, |
| | | Pathway | | signaling | | | | | 57498 | PSEN2, RPS6KA3, KIDINS220 |
| | | | | pathway | | | | | | |
| | 9_Summary | KEGG | ko04666 | Fc gamma R- | −2.54486 | 2.54486 | −0.960 | 6/91 | 1794, 3984, 5580, 10095, 27040, 50807 | DOCK2, LIMK1, PRKCD, ARPC1B, LAT, |
| | | Pathway | | mediated | | | | | | ASAP1 |
| | | | | phagocytosis | | | | | | |
| | 10_Summary | KEGG | ko05168 | Herpes simplex | −2.0881 | 2.088099 | −0.643 | 8/185 | 1936, 3150, 5371, 6428, 6672, 8683, | EEF1D, HMGN1, PML, SRSF3, SP100, |
| | | Pathway | | infection | | | | | 9641, 57506 | SRSF9, IKBKE, MAVS |
| | 11_Summary | KEGG | hsa04928 | parathyroid | −2.08798 | 2.087977 | −0.643 | 6/113 | 673, 1385, 4205, 4666, 9826, 11214 | BRAF, CREB1, MEF2A, NACA, ARHGEF11, |
| | | Pathway | | hormone | | | | | | AKAP13 |
| | | | | synthesis, | | | | | | |
| | | | | secretion and | | | | | | |
| | | | | action | | | | | | |
| RE- | 1_Summary | Reactome | R-HSA- | mRNA Splicing - | −12.9432 | 12.94322 | −9.568 | 22/183 | 3181, 3188, 4670, 4686, 6428, 6434, | HNRNPA2B1, HNRNPH2, HNRNPM, NCBP1, |
| ACTOME | | Gene | 72163 | Major Pathway | | | | | 8106, 8189, 8683, 9416, 10250, 10421, | SRSF3, TRA2B, PABPN1, SYMPK, |
| | | Sets | | | | | | | 10569, 10772, 11325, 22938, 23451, | SRSF9, DDX23, SRRM1, CD2BP2, SLU7, |
| | | | | | | | | | 23524, 27316, 51585, 55749, 199746, | SRSF10, DDX42, SNW1, SF3B1, SRRM2, |
| | | | | | | | | | 5903, 677, 1022, 1207, 5580, 6189, | RBMX, PCF11, CCAR1, U2AF1L4, |
| | | | | | | | | | 10528, 10785, 51602, 54888, 54913, | RANBP2, ZFP36L1, CDK7, CLNS1A, |
| | | | | | | | | | 55623, 55802, 85456, 255967 | PRKCD, RPS3A, NOP56, WDR4, NOP58, |
| | | | | | | | | | | NSUN2, RPP25, THUMPD1, DCP1A, |
| | | | | | | | | | | TNKS1BP1, PAN3 |
| | 2_Summary | Reactome | R-HSA- | Signaling by | −11.2138 | 11.2138 | −8.441 | 31/455 | 394, 695, 752, 1062, 1778, 2316, 3895, | ARHGAP5, BTK, FMNL1, CENPE, |
| | | Gene | 194315 | Rho GTPases | | | | | 3984, 4650, 4690, 5580, 5903, 6280, | DYNC1H1, FLNA, KTN1, LIMK1, MYO9B, |
| | | Sets | | | | | | | 6453, 7074, 8359, 9181, 9212, 9826, | NCK1, PRKCD, RANBP2, S100A9, ITSN1, |
| | | | | | | | | | 9938, 10095, 10928, 11214, 23380, | TIAM1, H4C1, ARHGEF2, AURKB, |
| | | | | | | | | | 23526, 51291, 55114, 57514, 64333, | ARHGEF11, ARHGAP25, ARPC1B, RALBP1, |
| | | | | | | | | | 79658, 94134 | AKAP13, SRGAP2, ARHGAP45, GMIP, |
| | | | | | | | | | | ARHGAP17, ARHGAP31, ARHGAP9, |
| | | | | | | | | | | ARHGAP10, ARHGAP12 |
| | 3_Summary | Reactome | R-HSA- | Transcriptional | −7.19094 | 7.190936 | −4.661 | 22/364 | 1022, 1025, 1108, 3065, 3066, 4361, | CDK7, CDK9, CHD4, HDAC1, HDAC2, |
| | | Gene | 3700989 | Regulation by | | | | | 4869, 5371, 6749, 6883, 7936, 8445, | MRE11, NPM1, PML, SSRP1, TAF12, |
| | | Sets | | TP53 | | | | | 8621, 8812, 9212, 9219, 10397, 23112, | NELFE, DYRK2, CDK13, CCNK, AURKB, |
| | | | | | | | | | 51755, 64121, 80196, 85456, 5253, | MTA2, NDRG1, TNRC6B, CDK12, RRAGC, |
| | | | | | | | | | 6601, 8359, 9611, 10943, 22992, 29072, | RNF34, TNKS1BP1, PHF2, SMARCC2, |
| | | | | | | | | | 64426, 221656, 10514, 23451, 1385, | H4C1, NCOR1, MSL3, KDM2A, SETD2, |
| | | | | | | | | | 5580, 8660, 10645 | SUDS3, KDM1B, MYBBP1A, SF3B1, |
| | | | | | | | | | | CREB1, PRKCD, IRS2, CAMKK2 |
| | 4_Summary | Reactome | R-HSA- | Vesicle- | −6.61081 | 6.610814 | −4.190 | 30/673 | 1062, 1778, 4253, 4644, 6453, 7109, | CENPE, DYNC1H1, MIA2, MYO5A, |
| | | Gene | 5653656 | mediated | | | | | 8578, 8867, 9135, 9685, 9847, 9919, | ITSN1, TRAPPC10, SCARF1, SYNJ1, |
| | | Sets | | transport | | | | | 9950, 10133, 10808, 22870, 23095, | RABEP1, CLINT1, C2CD5, SEC16A, GOLGA5, |
| | | | | | | | | | 23216, 23256, 26000, 26130, 27183, | OPTN, HSPH1, PPP6R1, KIF1B, TBC1D1, |
| | | | | | | | | | 51429, 55667, 55770, 57679, 83696, | SCFD1, TBC1D10B, GAPVD1, VPS4A, |
| | | | | | | | | | 84062, 84315, 85021 | SNX9, DENND4C, EXOC2, ALS2, TRAPPC9, |
| | | | | | | | | | | DTNBP1, MON1A, REPS1 |
| | 5_Summary | Reactome | R-HSA- | p75 NTR | −6.51168 | 6.511679 | −4.137 | 11/97 | 3065, 3066, 5664, 6453, 6868, 7074, | HDAC1, HDAC2, PSEN2, ITSN1, ADAM17, |
| | | Gene | 193704 | receptor- | | | | | 8878, 9181, 9826, 11214, 57142, 695, | TIAM1, SQSTM1, ARHGEF2, ARHGEF11, |
| | | Sets | | mediated | | | | | 3984, 10095 | AKAP13, RTN4, BTK, LIMK1, ARPC1B |
| | | | | signalling | | | | | | |
| | 6_Summary | Reactome | R-HSA- | Regulation of | −6.22875 | 6.228752 | −3.895 | 7/33 | 1385, 3065, 3066, 4204, 9212, 9611, | CREB1, HDAC1, HDAC2, MECP2, AURKB, |
| | | Gene | 9022692 | MECP2 | | | | | 23112, 1025, 3726, 8812, 22938, 5664, | NCOR1, TNRC6B, CDK9, JUNB, CCNK, |
| | | Sets | | expression and | | | | | 6868, 7090, 8359 | SNW1, PSEN2, ADAM17, TLE3, H4C1 |
| | | | | activity | | | | | | |
| | 7_Summary | Reactome | R-HSA- | SUMO E3 | −6.00854 | 6.00854 | −3.780 | 14/182 | 1069, 3065, 3066, 4869, 5371, 5903, | CETN2, HDAC1, HDAC2, NPM1, PML, |
| | | Gene | 3108232 | ligases | | | | | 6294, 6672, 7158, 8359, 9212, 9641, | RANBP2, SAFB, SP100, TP53BP1, H4C1, |
| | | Sets | | SUMOylate | | | | | 10155, 51602, 1385, 1778, 9611, 10951, | AURKB, IKBKE, TRIM28, NOP58, CREB1, |
| | | | | target proteins | | | | | 27183 | DYNC1H1, NCOR1, CBX1, VPS4A |
| | 8_Summary | Reactome | R-HSA- | Signaling by | −5.11633 | 5.116326 | −2.946 | 11/134 | 673, 1108, 1385, 2889, 3726, 4205, | BRAF, CHD4, CREB1, RAPGEF1, JUNB, |
| | | Gene | 166520 | NTRKs | | | | | 4665, 6197, 7074, 8660, 57498, 4670, | MEF2A, NAB2, RPS6KA3, TIAM1, IRS2, |
| | | Sets | | | | | | | 4686, 4690, 5580, 5664, 6696, 6868, | KIDINS220, HNRNPM, NCBP1, NCK1, |
| | | | | | | | | | 9611, 64759 | PRKCD, PSEN2, SPP1, ADAM17, NCOR1, |
| | | | | | | | | | | TNS3 |
| | 9_Summary | Reactome | R-HSA- | Diseases of | −4.43377 | 4.433767 | −2.356 | 18/387 | 673, 1385, 3065, 3066, 4000, 4296, | BRAF, CREB1, HDAC1, HDAC2, LMNA, |
| | | Gene | 5663202 | signal | | | | | 4686, 5664, 6868, 7094, 8660, 9208, | MAP3K11, NCBP1, PSEN2, ADAM17, |
| | | Sets | | transduction by | | | | | 9611, 22938, 27044, 51411, 56829, | TLN1, IRS2, LRRFIP1, NCOR1, SNW1, |
| | | | | growth factor | | | | | 399687, 6734, 7709 | SND1, BIN2, ZC3HAV1, MYO18A, SRPRA, |
| | | | | receptors and | | | | | | ZBTB17 |
| | | | | second | | | | | | |
| | | | | messengers | | | | | | |
| | 10_Summary | Reactome | R-HSA- | Formation of | −4.38067 | 4.380666 | −2.356 | 7/61 | 1022, 1025, 4686, 6749, 7936, 8812, | CDK7, CDK9, NCBP1, SSRP1, NELFE, |
| | | Gene | 112382 | RNA Pol II | | | | | 27125, 6883, 8621, 51755, 5451, | CCNK, AFF4, TAF12, CDK13, CDK12, |
| | | Sets | | elongation | | | | | 23248, 51585, 5903, 27183, 1794, | POU2F1, RPRD2, PCF11, RANBP2, VPS4A, |
| | | | | complex | | | | | 4869 | DOCK2, NPM1 |
| | 11_Summary | Reactome | R-HSA- | Apoptotic | −3.84195 | 3.84195 | −2.056 | 6/52 | 1676, 3008, 4000, 5580, 7431, 22985 | DFFA, H1-4, LMNA, PRKCD, VIM, ACIN1 |
| | | Gene | 75153 | execution | | | | | | |
| | | Sets | | phase | | | | | | |
| | 12_Summary | Reactome | R-HSA- | Initiation of | −3.74765 | 3.747653 | −2.006 | 4/19 | 4000, 22933, 23141, 23592, 984, 1022, | LMNA, SIRT2, ANKLE2, LEMD3, CDK11B, |
| | | Gene | 2995383 | Nuclear | | | | | 1062, 1069, 1778, 3065, 4171, 4361, | CDK7, CENPE, CETN2, DYNC1H1, HDAC1, |
| | | Sets | | Envelope (NE) | | | | | 4869, 5903, 5925, 7158, 8359, 9212, | MCM2, MRE11, NPM1, RANBP2, RB1, |
| | | | | Reformation | | | | | 10051, 10133, 11083, 27183, 64151 | TP53BP1, H4C1, AURKB, SMC4, OPTN, |
| | | | | | | | | | | DIDO1, VPS4A, NCAPG |
| | 13_Summary | Reactome | R-HSA- | RHO GTPase | −3.72629 | 3.726292 | −1.996 | 15/327 | 695, 752, 1062, 1778, 2316, 3895, | BTK, FMNL1, CENPE, DYNC1H1, FLNA, |
| | | Gene | 195258 | Effectors | | | | | 3984, 4690, 5580, 5903, 6280, 8359, | KTN1, LIMK1, NCK1, PRKCD, RANBP2, |
| | | Sets | | | | | | | 9212, 10095, 23380, 4644, 4650 | S100A9, H4C1, AURKB, ARPC1B, |
| | | | | | | | | | | SRGAP2, MYO5A, MYO9B |
| | 14_Summary | Reactome | R-HSA- | Nervous | −3.15123 | 3.151235 | −1.569 | 20/577 | 1385, 3066, 3984, 4478, 4650, 4665, | CREB1, HDAC2, LIMK1, MSN, MYO9B, |
| | | Gene | 9675108 | system | | | | | 4686, 4690, 5664, 6189, 6197, 6453, | NAB2, NCBP1, NCK1, PSEN2, RPS3A, |
| | | Sets | | development | | | | | 7074, 7094, 8660, 9826, 10095, 22885, | RPS6KA3, ITSN1, TIAM1, TLN1, IRS2, |
| | | | | | | | | | 23380, 57698 | ARHGEF11, ARPC1B, ABLIM3, SRGAP2, |
| | | | | | | | | | | SHTN1 |
| | 15_Summary | Reactome | R-HSA- | Prolonged ERK | −2.93454 | 2.934538 | −1.429 | 3/14 | 673, 2889, 57498, 8660, 23533 | BRAF, RAPGEF1, KIDINS220, IRS2, |
| | | Gene | 169893 | activation | | | | | | PIK3R5 |
| | | Sets | | events | | | | | | |
| | 16_Summary | Reactome | R-HSA- | TRAF3- | −2.93454 | 2.934538 | −1.429 | 3/14 | 7706, 9641, 57506 | TRIM25, IKBKE, MAVS |
| | | Gene | 918233 | dependent IRF | | | | | | |
| | | Sets | | activation | | | | | | |
| | | | | pathway | | | | | | |
| | 17_Summary | Reactome | R-HSA- | Estrogen- | −2.63268 | 2.632685 | −1.192 | 8/150 | 1025, 3065, 5451, 5469, 7090, 8359, | CDK9, HDAC1, POU2F1, MED1, TLE3, |
| | | Gene | 9018519 | dependent | | | | | 9166, 23112, 1385, 9611, 221656 | H4C1, EBAG9, TNRC6B, CREB1, NCOR1, |
| | | Sets | | gene | | | | | | KDM1B |
| | | | | expression | | | | | | |
| | 18_Summary | Reactome | R-HSA- | Interferon | −2.54486 | 2.54486 | −1.129 | 6/91 | 960, 3383, 5371, 5580, 6672, 7706, | CD44, ICAM1, PML, PRKCD, SP100, |
| | | Gene | 877300 | gamma | | | | | 2316, 5903, 25939 | TRIM25, FLNA, RANBP2, SAMHD1 |
| | | Sets | | signaling | | | | | | |
| | 19_Summary | Reactome | R-HSA- | Neutrophil | −2.46537 | 2.465365 | −1.059 | 16/480 | 728, 960, 1778, 1794, 3614, 3615, | C5AR1, CD44, DYNC1H1, DOCK2, |
| | | Gene | 6798695 | degranulation | | | | | 3689, 5223, 5315, 5580, 6280, 8621, | IMPDH1, IMPDH2, ITGB2, PGAM1, PKM, |
| | | Sets | | | | | | | 9961, 23526, 51411, 64333 | PRKCD, S100A9, CDK13, MVP, ARHGAP45, |
| | | | | | | | | | | BIN2, ARHGAP9 |
| | 20_Summary | Reactome | R-HSA- | COPII- | −2.40021 | 2.400209 | −1.017 | 5/68 | 7109, 9919, 22870, 23256, 83696 | TRAPPC10, SEC16A, PPP6R1, SCFD1, |
| | | Gene | 204005 | mediated | | | | | | TRAPPC9 |
| | | Sets | | vesicle | | | | | | |
| | | | | transport | | | | | | |

1. A specialized pro-resolving mediator (SPM) or derivative thereof for use in treating or preventing an inflammatory condition or disease.

2. A cell for use in a method of treating or preventing an inflammatory condition or disease, wherein the method comprises the step of contacting a monocyte or monocyte-derived macrophage (MDM) with a specialized pro-resolving mediator (SPM) or derivative thereof to obtain the cell.

3. The SPM or cell for use according to claim 1 or 2, wherein the inflammatory condition or disease is rheumatoid arthritis (RA), osteoarthritis or atherosclerosis.

4. The SPM or cell for use according to any preceding claim, wherein the SPM is selected from the group consisting of a maresin, a protectin, a resolvin and an E-series resolvin.

5. The SPM or cell for use according to any preceding claim, wherein the SPM is a maresin conjugate in tissue regeneration (MCTR).

6. The SPM or cell for use according to any preceding claim, wherein the SPM is selected from the group consisting of MCTR3, MCTR1 and MCTR2, optionally wherein the SPM is MCTR3.

7. The cell for use according to any one of claims 2-5, wherein the cell is a monocyte-derived macrophage (MDM).

8. The SPM or cell for use according to any preceding claim, wherein (a) joint inflammation is reduced or prevented; and/or (b) bone and/or cartilage is repaired or protected.

9. The SPM or cell for use according to any preceding claim, wherein the SPM or cell is administered to a subject after RA onset.

10. The SPM or cell for use according to any preceding claim, wherein the SPM or cell is administered during arthritic inflammation.

11. The SPM or cell for use according to any preceding claim, wherein the SPM or cell is administered intravenously or intra-articularly.

12. A method of culturing a population of monocytes or monocyte-derived macrophages (MDMs), the method comprising:
(a) providing a population of monocytes or MDMs; and
(b) contacting the population of monocytes or MDMs with a specialized pro-resolving mediator (SPM) or derivative thereof.

13. The method of claim 12, wherein the SPM is selected from the group consisting of a maresin, a protectin, a resolvin and an E-series resolvin.

14. The method of claim 12 or 13, wherein the SPM is a maresin conjugate in tissue regeneration (MCTR).

15. The method of any one of claims 12-14, wherein the SPM is selected from the group consisting of MCTR3, MCTR1 and MCTR2, optionally wherein the SPM is MCTR3.

16. The method of any one of claims 12-15, wherein monocytes are differentiated to monocyte-derived macrophages (MDMs).

17. The method of any one of claims 12-16, wherein the population of monocytes is contacted with the SPM or derivative thereof for 1-72 hours.

18. The method of any one of claims 12-17, wherein the SPM or derivative thereof is contacted with the population of monocytes at a final concentration of 0.001-100 nM.

19. A population of cells obtainable by the method of any one of claims 12-18.

20. A pharmaceutical composition comprising the population of cells of claim 19 and a pharmaceutically-acceptable carrier, excipient and/or diluent.

21. The population of cells of claim 19 or pharmaceutical composition of claim 20 for use in treating or preventing an inflammatory condition or disease, optionally wherein the inflammatory condition or disease is rheumatoid arthritis (RA), osteoarthritis or atherosclerosis.

22. The cell for use according to any one of claims 2-11 or the method of any one of claims 12-18, wherein the contacting with the SPM or derivative thereof increases expression of arginase-1 (Arg-1), interleukin-10 (IL-10), Dbl1 and/or TGFb.

23. The cell for use according to any one of claims 2-11, the population of cells of claim 19, the pharmaceutical composition of claim 20, or the population of cells or pharmaceutical composition for use according to claim 21, wherein the cells are Arg1+, IL-10+, DBI1+ and/or TGFb+.

24. A method of treating or preventing an inflammatory condition or disease comprising administering a specialized pro-resolving mediator (SPM) or derivative thereof to a subject in need thereof, optionally wherein the inflammatory condition or disease is rheumatoid arthritis (RA), osteoarthritis or atherosclerosis.

25. A method of treating or preventing an inflammatory condition or disease comprising the steps:
(a) providing a population of monocytes or monocyte-derived macrophages (MDMs);
(b) contacting the population of monocytes or MDMs with a specialized pro-resolving mediator (SPM) or derivative thereof; and
(c) administering the cells from step (b) or a portion thereof to a subject in need thereof;
optionally wherein the inflammatory condition or disease is rheumatoid arthritis (RA), osteoarthritis or atherosclerosis.

26. A method of diagnosing an inflammatory condition or disease comprising the steps:
(a) determining the level of one or more maresin conjugate in tissue regeneration (MCTR) in one or more plasma sample obtained from a subject;
(b) comparing the level of the one or more MCTR to one or more corresponding reference value;
wherein the level of the one or more MCTR compared to the one or more reference value is indicative of the inflammatory condition or disease, optionally wherein the inflammatory condition or disease is rheumatoid arthritis (RA), osteoarthritis or atherosclerosis.
